(19) United States
(12) Patent Application Publication
(10) Pub. No.: US 2006/0191037 A1
(43) Pub. Date: Aug. 24, 2006

(54) Plant transcription factor that is involved in sugar signalling

(76) Inventors: Christer Jansson, Vallentuna (SE); Chuanxin Sun, Uppsala (SE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/537,035

(22) PCT Filed: Dec. 2, 2003
(86) PCT No.: PCT/GB03/05245

§ 371 Date: Oct. 12, 2005

(30) Foreign Application Priority Data
Dec. 2, 2002 (GB) 0228094.9
Sep. 24, 2003 (GB) 0322408.6

(51) Int. Cl. A01H 1/00; C12Q 1/68; C07H 21/04; C08B 31/00; C12N 5/04

(52) U.S. Cl. 800/284; 435/6; 435/419; 435/468; 536/23.2; 536/102

(57) ABSTRACT

Provided are nucleotide sequences which encodes a sugar-signalling transcription factors capable of activating a promoter of a gene encoding an enzyme involved in the synthesis or deposition of starch e.g. in response to sugar levels in a plant. Preferred sequences encode WRKY proteins such as the sugar signaling in barley2 (SUSIBA2) factor and variants thereof, and act on promoters which comprise at least one SURE element and\or W box element to which the transcription factor binds (e.g. promoters such as iso1, sbe1, sbeIIb, ssI, agpaseS). The invention also provides related methods and materials e.g. for activating, modulating, and investigating the elements present in such promoters.

### TECHNICAL FIELD

The present invention relates generally to methods and materials based on transcription factors involved in sugar signalling in plants.

### BACKGROUND ART

Development of the cereal seed is orchestrated by the coordinated activities of a large number of genes that encode metabolic and regulatory enzymes, as well as other proteins (Olsen, 2001). This results in a triploid endosperm, the embryo, pericarp, seed coat and other tissues of the mature grain. The endosperm structure consists of two tissues, the interior starch-filled endosperm and the outer epidermal layer called the aleurone.

Starch, which is a mixture of amylopectin (a heavily branched polyglucan) and amylose (a mostly linear polyglucan), is deposited in the endosperm as granules. The synthesis and deposition of starch in the endosperm depend on enzymes such as ADP-glucose pyrophosphorylase (AGPase), starch synthases (SS), starch branching enzymes (SBE), and starch debranching enzymes (DBE). Most, if not all, of these enzymes exist in two or more isoforms (see Ball et al., 1998; Buleon et al., 1998; Myers et al., 2000; Nakamura, 2002; Smith, 2001; for recent reviews on starch biosynthesis). DBEs are grouped into two distinct classes, isoamylase and pullulanase each with different isoforms (Nakamura, 1996). Isoamylase (EC 3.2.1.68) is an essential enzyme in amylopectin synthesis. However, the precise role of the enzyme in this process is still not clear, and different models have been proposed (Ball et al., 1998; Nakamura, 2002; Smith, 2001).

It has been reported that expression of starch synthesis genes, i.e. SS in potato (Visser et al., 1991), ADPGase in potato (Müller-Röber et al., 1990), sweet potato (Bae and Liu, 1997), Arabidopsis (Rook et al., 2001) and tomato (Li et al, 2002), SBE in potato (Kossman et al., 1991), maize (Kim and Guiltinan, 1999) and Arabidopsis (Khoshnoodi et al., 1998), and isoamylase in barley (Sun et al., 1999), are sugar inducible. In contrast to the situation in bacteria, yeast and mammals, where sugar signaling cascades are extensively studied, the sugar signaling transduction pathways in plants are poorly understood (Rolland et al., 2002). Generally, in higher plants, high sugar levels stimulate expression of genes involved in sink function, such as growth, storage of proteins and biosynthesis of starch and other carbohydrates, whereas low sugar levels promote photosynthesis and mobilization of energy reserves, such as breakdown of storage starch or lipids.

Sugar signaling can be dissected into three steps, sugar sensing, signal transduction, and target gene expression. The picture is clouded by the dual function of sugars as nutrients and signaling molecules, and by the interaction (in plants and animals) between sugar signaling and hormonal networks. In plants the complexity is further increased by the vital role of sugar production through photosynthesis. Hexoses, sucrose and trehalose might serve as elicitors of plant sugar signaling (Goddijn and Smeekens, 1998; Rolland et al., 2002). Hexokinase, sucrose and glucose transporters, and various sugar receptors, have been proposed as components of the sugar sensing machinery (Rolland et al., 2002; Sheen et al., 1999; Smeekens, 2000).

The Ser/Thr protein kinase Snf1 is a central participant in yeast sugar signaling (Carlson, 1999). Snf1 phosphorylates downstream components and is also itself activated by phosphorylation. Snf related protein kinases (SnRKs) are found in yeast, mammals and plants, where they partake in a large number of regulatory functions (Halford and Hardie, 1998; Hardie et al., 1998). There is evidence that some plant SnRKs share functional homology with Snf1 in plant sugar signaling, although the exact nature of their responses to sugars remains to be clarified (Rolland et al., 2002). Other players implicated in sugar signaling transduction pathways in plants are sugar metabolites, 14-3-3 proteins, trehalose-6-phosphate, and Ca²⁺ (Rolland et al., 2002).

Little is known about the cis and trans factors mediating the final steps in plant sugar signaling. To date, five different types of cis elements have been identified in sugar-regulated plant promoters; the SURE (Grierson et al., 1994), SP8 (Ishiguro and Nakamura, 1994), TGGACGG (Maeo et al., 2001), G box (Giuliano et al., 1988) and B box (Grierson et al., 1994; Zourelidou et al., 2002) elements.

Only two putative transcription factors, SPF1 and STK, with relevance to plant sugar signaling have been isolated. SPF1 was isolated from sweet potato, and it was reported that SPF1 binds to the SP8a and SP8b promoter elements of the β-amylase and sporamin genes of tuberous roots, where it functions as a repressor (Ishiguro and Nakamura, 1994). STK binds to the B box as an activator (Zourelidou et al., 2002).

It can thus be seen that novel transcription factors, for instance those which could be used to affect starch synthesis or deposition (and hence structure) may provide a useful contribution to the art.

### DISCLOSURE OF THE INVENTION

The present inventors have isolated a transcription factor, SUSIBA2 (sugar signaling in barley), and examined its interaction with endosperm specific expression of the sbeIIb (encoding SBEIIb) and iso1 (encoding isoamylase 1) genes during barley seed development.

Antibodies against SUSIBA2 were produced and the expression pattern for susiba2 was determined at the RNA and protein levels. It was found that susiba2 is expressed in endosperm but not leaves. Transcription of susiba2 is sugar inducible and ectopic susiba2 expression was obtained in sugar-treated leaves. The temporal expression of susiba2 in barley endosperm followed that of iso1 and endogenous sucrose levels, with a peak at around 12 days after pollination.

It was previously known that barley iso1 harbors an SP8a element and that it contributes to the endosperm specificity of iso1 expression by recruiting a repressor in non-expressing tissues (Sun et al., 1999). Similarly, the endosperm-specific expression of barley sbeIIb (Sun et al., 1998) is partly determined by a repressor-binding B box element, Bbl, in the second intron of the gene (Ahlandsberg et al., 2002b).

Interestingly SUSIBA2 appears to bind, as an activator, to the SURE (sugar responsive) element in the iso1 promoter. It also binds the W box elements but not to the SP8a element. A novel application of a transcription factor oligodeoxynucleotide decoy strategy with transformed barley endosperm was used to provide experimental evidence for the importance of the SURE element in iso1 transcription.

Thus, not only is its biding specificity different to SPF1 and STK described above, but SUSIBA2 also represents the first isolated SURE-binding transcription factor, the first WRKY protein known to be involved in carbohydrate anabolism, and the first transcription factor of any sort reported for regulation of starch synthesis. It shares only a very low degree of sequence identity (28%) with SPF1. Orthologs to SUSIBA2 were also isolated from rice and wheat endosperm.

The sequences from the rice and wheat cDNAs, and wheat peptide are annexed hereto.

Thus in a first aspect of the invention there is disclosed an isolated nucleic acid which comprises, or consists essentially of, a nucleotide sequence which encodes a transcription factor which is capable of modulating the activity of a promoter of a gene encoding an enzyme involved in the synthesis or deposition of starch.

“Nucleic acid” and “nucleic acid molecule” have the same meaning. As stated above, the nucleic acid may consist essentially of a nucleotide sequence of the present invention (which is to say that the sequence is ‘of the essence of’ the molecule, generally making up more than 50% of it).

The promoter will preferably include at least one SURE element (as described in more detail hereinafter—see Example 7 in particular) and\or W box element and the transcription factor will bind to one or more of these.

The enzyme (and hence promoter) may optionally be selected from the following genes: iso1, sbe1, sbeIIb, ssI, agpaseS.

Preferably the transcription factor is a WRKY protein which is involved in carbohydrate anabolism. More specifically it modulates the activity of the promoter within a plant in response to sugar levels in the plant i.e. the factor is a component of a sugar signaling pathway which is responsible for modulating target gene expression.

Preferably the nucleotide sequence is a SUSIBA2 nucleotide sequence which encodes the 573-amino acid sequence given in FIG. 1.

Preferably the nucleotide sequence is a SUSIBA2 nucleotide sequence which consists of the coding sequence given in FIG. 1 or one which is degeneratively equivalent or complementary thereto. The length of the susiba2 cDNA is 2355 nucleotides, and the open reading frame starts at position 247 and ends at position 1966 (FIG. 1).

In alternative embodiments the nucleotide sequence is a SUSIBA2 nucleotide sequence which encodes an amino acid sequence given in the Sequence Annex. In preferred alternative embodiments the nucleotide sequence is a SUSIBA2 nucleotide sequence which comprises a coding sequence given in the Sequence Annex or one which is degeneratively equivalent or complementary thereto. Where aspects or embodiments of the invention are discussed in relation to the barley sequence given in FIG. 1, it will be understood that each of those aspects or embodiments applies mutatis mutandis to the wheat or rice orthologues, as described in the Sequence Annex.

The nucleic acid molecules or vectors (see below) according to the present invention may be provided isolated and/or purified from their natural environment, in substantially pure or homogeneous form, or free or substantially free of nucleic acid or genes of the species of interest or origin other than the sequence encoding a polypeptide with the required function. The term “isolated” encompasses all these possibilities.

Nucleic acid according to the present invention may include cDNA, RNA, genomic DNA and modified nucleic acids or nucleic acid analogs. Where a DNA sequence is specified, e.g. with reference to a figure, unless context requires otherwise the RNA equivalent, with U substituted for T where it occurs, is encompassed.

Complement sequences of those discussed herein are also encompassed. As is well understood by those skilled in the art, two nucleic acid nucleotide sequences are “complementary” when one will properly base pair with all or part of the other according to the standard rules (G pairs with C, and A pairs with T). One sequence is “the complement” of another where those sequences are of the same length, but are complementary to each other.

In a further aspect of the present invention there is disclosed a nucleic acid molecule comprising (or preferably consisting essentially of) a nucleotide sequence which is a variant of the barley SUSIBA2 nucleotide sequence of FIG. 1.

Thus the invention provides, inter alia, an isolated transcription factor gene derivable from (and which can be expressed recombinantly in) maturing seeds, and which encodes a transcription factor protein which targets a promoter of a gene encoding an enzyme involved in the synthesis or deposition (anabolism) of starch. Such genes may encode SUSIBA2, or are related or derived therefrom e.g. genes which encode an RNA which hybridizes to the SUSIBA2 gene under high stringency conditions. The transcription factors may bind a SURE and\or W box element in the promoter, and optionally not bind the SP8a element.

The gene may be present in an expression cassette, and the transcription factor of the invention may be used, for example, in a method for enhancing or reducing expression of a starch anabolic enzyme, which method may involve transforming a seed crop plant with the factor gene, said plant thereby expressing the transcription factor protein encoded by said transcription factor gene. This may be used to give enhanced, reduced, or altered starch synthesis. More specifically the disclosure of these sequences provides a novel mechanism for manipulating the starch anabolism activity in plants in a number of important respects. These include, inter alia, the ability to: modulate the activity of isoamylases and other enzymes in plants; alter responsiveness of this pathway to sugar levels; alter overall debranching enzyme activity; alter such enzyme activities in various different tissues or subcellular compartments or at various different developmental stages and produce novel starch types in transgenic line.

Some aspects and embodiments of the invention will now be discussed in more detail.

SUSIBA2 Variants

Variants of the present invention can be artificial nucleic acids (i.e. containing sequences which have not originated naturally) which can be prepared by the skilled person in the light of the present disclosure. Artificial variants (derivatives) may be prepared by those skilled in the art, for instance by site directed or random mutagenesis, or by direct synthesis. Preferably the variant nucleic acid is generated either directly or indirectly (e.g. via one or amplification or replication steps) from an original nucleic acid having all or part of the sequences of the first aspect. Preferably the variant encodes a product which has one or more of the transcription factor activities discussed above.

Alternatively they may be novel, naturally occurring, nucleic acids, isolatable using the sequences of the present invention. Sequence variants which occur naturally may also include alleles (which will include polymorphisms or mutations at one or more bases). Preferred nucleic acids are those encoding orthologs from any of rice, wheat, or potato or derivatives of these as described in more detail below.

Thus a variant may be or include a distinctive part or fragment (however produced) corresponding to a portion of the sequence provided. These portions may include motifs which are distinctive to SUSIBA2 sequences, such motifs being discussed in more detail in the Examples below.

Fragments may encode or omit particular functional parts of the polypeptide. Equally the fragments may have utility in probing for, or amplifying, the sequence provided or closely related ones. Suitable lengths of fragment, and conditions, for such processes are discussed in more detail below. Also included are nucleic acids which have been extended at the 3′ or 5′ terminus with respect to those of the first aspect.

The term “variant” nucleic acid as used herein encompasses all of these possibilities. When used in the context of polypeptides or proteins it indicates the encoded expression product of the variant nucleic acid.

Some of the aspects of the present invention relating to variants will now be discussed in more detail.

Homology (either similarity or identity) may be as defined and determined by the TBLASTN program, of Altschul et al. (1990) J. Mol. Biol. 215: 403-10, which is in standard use in the art, or, and this may be preferred, the standard program BestFit, which is part of the Wisconsin Package, Version 8, September 1994, (Genetics Computer Group, 575 Science Drive, Madison, Wis., USA, Wisconsin 53711). BestFit makes an optimal alignment of the best segment of similarity between two sequences. Optimal alignments are found by inserting gaps to maximize the number of matches using the local homology algorithm of Smith and Waterman.

Homology, with respect to SUSIBA2 in FIG. 1 may be at the nucleotide sequence and/or encoded amino acid sequence level. Preferably, the nucleic acid and/or amino acid sequence shares at least about 50%, or 60%, or 70%, or 80% homology, most preferably at least about 90%, 95%, 96%, 97%, 98% or 99% similarity or identity.

Thus a variant polypeptide in accordance with the present invention may include within an amino acid sequences described herein a single amino acid or 2, 3, 4, 5, 6, 7, 8, or 9 changes, about 10, 15, 20, 30, 40 or 50 changes, or greater than about 50, 60, 70, 80, 90, 100 or 115 changes. In addition to one or more changes within the amino acid sequence shown, a variant polypeptide may include additional amino acids at the C-terminus and/or N-terminus.

Thus in a further aspect of the invention there is disclosed a method of producing a derivative nucleic acid comprising the step of modifying the coding sequence of a nucleic acid comprising any one the sequences discussed above.

Changes to a sequence, to produce a derivative, may be by one or more of addition, insertion, deletion or substitution of one or more nucleotides in the nucleic acid, which may lead to the addition, insertion, deletion or substitution of one or more amino acids in the encoded polypeptide. Changes may be desirable for a number of reasons, including introducing or removing the following features: restriction endonuclease sequences; codon usage; other sites which are required for post translation modification; cleavage sites in the encoded polypeptide; motifs in the encoded polypeptide (e.g. binding sites). Leader or other targeting sequences (e.g. the putative TM region) may be added or removed from the expressed protein to determine its location following expression. All of these may assist in efficiently cloning and expressing an active polypeptide in recombinant form (as described below).

Other desirable mutation may be random or site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered stability or specificity.

Other methods for generating novel specificities may include mixing or incorporating sequences from related SUSIBA genes into the sequences disclosed herein. For example restriction enzyme fragments of related genes could be ligated together. An alternative strategy for modifying SUSIBA2 sequences would employ PCR as described below (Ho et al., 1989, Gene 77, 51-59) or DNA shuffling (Crameri et al., 1998, Nature 391).

In a further aspect of the present invention there is provided a method of detecting, identifying and/or cloning (isolating) a nucleic acid of the present invention (e.g. a homologue of the sequences set out hereinafter) from a plant which method employs any of the sequences of the invention discussed above. In particular the methods will generally employ primers or probes derived from all or part of these sequences (or sequences complementary thereto) set out herein.

An oligonucleotide primer for use in amplification reactions may be about 30 or fewer nucleotides in length. Generally specific primers are upwards of 12, 13, 14, 15, 18, 21 or 24 nucleotides in length. For optimum specificity and cost effectiveness, primers of 16-24 nucleotides in length may be preferred.

An oligonucleotide or polynucleotide probe may be based on the any of the sequences disclosed herein (e.g. introns or exons, although the latter may be preferred). If required, probing can be done with entire restriction fragments of the genes which may be 100's or even 1000's of nucleotides in length.

Those skilled in the art are well versed in the design of primers for use processes such as PCR. The primers will usually be based on sequences which are peculiar or unique to the SUSIBA2 sequences (see e.g. discussion of C-terminal sequence in the Examples below).

When using such probes or primers, if need be, clones or fragments identified in the search can be extended. For instance if it is suspected that they are incomplete, the original DNA source (e.g. a clone library, mRNA preparation etc.) can be revisited to isolate missing portions e.g. using sequences, probes or primers based on that portion which has already been obtained to identify other clones containing overlapping sequence.

In one embodiment, a variant in accordance with the present invention is also obtainable by means of a method which includes:

(a) providing a preparation of nucleic acid, e.g. from plant cells,

(b) providing a probe or primer as discussed above,

(c) contacting nucleic acid in said preparation with said nucleic acid molecule under conditions for hybridisation of said nucleic acid molecule to any said gene or homologue in said preparation, and identifying said gene or homologue if present by its hybridisation with said nucleic acid molecule.

Plants which may be a suitable source of SUSIBA2 may include any cereal, and in particular the developing seeds of such cereals.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as is described hereinafter. Probing may optionally be done by means of so-called “nucleic acid chips” (see Marshall & Hodgson (1998) Nature Biotechnology 16: 27-31, for a review).

Preliminary experiments may be performed by hybridising under low stringency conditions. For probing, preferred conditions are those which are stringent enough for there to be a simple pattern with a small number of hybridisations identified as positive which can be investigated further.

For instance, screening may initially be carried out under conditions, which comprise a temperature of about 37° C. or less, a formamide concentration of less than about 50%, and a moderate to low salt (e.g. Standard Saline Citrate (“SSC”)=0.15 M sodium chloride; 0.15 M sodium citrate; pH 7) concentration.

Alternatively, a temperature of about 50° C. or less and a high salt (e.g. “SSPE” 0.180 mM sodium chloride; 9 mM disodium hydrogen phosphate; 9 mM sodium dihydrogen phosphate; 1 mM sodium EDTA; pH 7.4). Preferably the screening is carried out at about 37° C., a formamide concentration of about 20%, and a salt concentration of about 5×SSC, or a temperature of about 50° C. and a salt concentration of about 2×SSPE. These conditions will allow the identification of sequences which have a substantial degree of homology (similarity, identity) with the probe sequence, without requiring the perfect homology for the identification of a stable hybrid.

Suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

It is well known in the art to increase stringency of hybridisation gradually until only a few positive clones remain. Suitable conditions would be achieved when a large number of hybridising fragments were obtained while the background hybridisation was low. Using these conditions nucleic acid libraries, e.g. cDNA libraries representative of expressed sequences, may be searched. Those skilled in the art are well able to employ suitable conditions of the desired stringency for selective hybridisation, taking into account factors such as oligonucleotide length and base composition, temperature and so on.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below) or RNAase cleavage. The identification of successful hybridisation is followed by isolation of the nucleic acid which has hybridised, which may involve one or more steps of PCR or amplification of a vector in a suitable host.

In a further embodiment, hybridisation of nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of are employed. Using RACE PCR, only one such primer may be needed (see “PCR protocols; A Guide to Methods and Applications”, Eds. Innis et al, Academic Press, New York, (1990)).

Thus a method involving use of PCR in obtaining nucleic acid according to the present invention may be carried out as described above, but using a pair of nucleic acid molecule primers useful in (i.e. suitable for) PCR.

The methods described above may also be used to determine the presence of one of the nucleotide sequences of the present invention within the genetic context of an individual plant, optionally a transgenic plant which may be produced as described in more detail below. This may be useful in plant breeding programmes e.g. to directly select plants containing alleles which are responsible for desirable traits in that plant species, either in parent plants or in progeny (e.g. hybrids, F1, F2 etc.). Thus use of the markers defined in the Examples below, or markers which can be designed by those skilled in the art on the basis the nucleotide sequence information disclosed herein, forms one part of the present invention.

As used hereinafter, unless the context demands otherwise, the term “SUSIBA2 nucleic acid” is intended to cover any of the nucleic acids of the invention described above, including functional variants.

SUSIBA2 Vectors and Transformation

In one aspect of the present invention, the SUSIBA2 nucleic acid described above is in the form of a recombinant and preferably replicable vector.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eucaryotic (e.g. higher plant, yeast or fungal cells).

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

Thus this aspect of the invention provides a gene construct, preferably a replicable vector, comprising a promoter operatively linked to a nucleotide sequence provided by the present invention.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press.

Many known techniques and protocols for manipulation of nucleic acid, for example in preparation of nucleic acid constructs, mutagenesis (see above discussion in respect of variants), sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

In one embodiment of this aspect of the present invention, there is provided a gene construct, preferably a replicable vector, comprising an inducible promoter operatively linked to a nucleotide sequence provided by the present invention.

The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Particular of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

The present invention also provides methods comprising introduction of such a construct into a host cell, particularly a plant cell.

In a further aspect of the invention, there is disclosed a host cell containing a heterologous nucleic acid or construct according to the present invention, especially a plant or a microbial cell.

The term “heterologous” is used broadly in this aspect to indicate that the SUSIBA2 nucleic acid in question has been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence.

Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homolog is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

The host cell (e.g. plant cell) is preferably transformed by the construct, which is to say that the construct becomes established within the cell, altering one or more of the cell's characteristics and hence phenotype e.g. with respect to sugar sensing or starch anabolism.

Nucleic acid can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species. Recently, there has also been substantial progress towards the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

Thus a further aspect of the present invention provides a method of transforming a plant cell involving introduction of a construct as described above into a plant cell and causing or allowing recombination between the vector and the plant cell genome to introduce a nucleic acid according to the present invention into the genome.

The invention further encompasses a host cell transformed with nucleic acid or a vector according to the present invention especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162; Vasil, et al. (1992) Bio/Technology 10, 667-674; Vain et al., 1995, Biotechnology Advances 13 (4): 653-671; Vasil, 1996, Nature Biotechnology 14 page 702).

Plants which include a plant cell according to the invention are also provided.

In addition to the regenerated plant obtainable by the above method, the present invention embraces all of the following: a clone of such a plant; selfed or hybrid progeny; descendants (e.g. F1 and F2 descendants) and any part of any of these. The invention also provides a plant propagule from such plants, that is any part which may be used in reproduction or propagation, sexual or asexual, including cuttings, and so on. In each case these embodiments will include a heterologous SUSIBA2 nucleic acid according to the present invention.

Uses of SUSIBA2 Nucleic Acids and Transcription Factors

The present invention provides a method of influencing the starch anabolism in a plant, the method including the step of causing or allowing expression of the product (polypeptide or nucleic acid transcript) encoded by heterologous nucleic acid according to the invention from that nucleic acid within cells of the plant.

The step may be preceded by the earlier step of introduction of the nucleic acid into a cell of the plant or an ancestor thereof.

Also provided is a method of binding, activating, or identifying a promoter which includes at least one SURE element and\or W box element, which method employs the step of contacting said promoter with a transcription factor as described herein. Where such method is performed in vivo, it will be a heterologous transcription factor.

Activation of a promoter may be confirmed by use of a reporter gene e.g. as described in the Examples herein.

Also provided is a method of identifying a sugar responsive element in a promoter, which method employs the step of contacting said promoter with a transcription factor as described herein. Where such method is performed in vivo, it will generally be a heterologous transcription factor. The contacting step is optionally followed by detection of binding to the promoter or region of the promoter. For example, elements within promoters may be characterised by use of EMSA or DNAase foot-printing techniques e.g. as described in the Examples herein.

The foregoing discussion has been generally concerned with uses of the nucleic acids of the present invention for production of functional SUSIBA2 polypeptides in a plant.

However the information disclosed herein may also be used to reduce the activity or levels of such polypeptides in cells in which it is desired to do so. For instance the sequence information disclosed herein may be used for the down-regulation of expression of genes e.g. using anti-sense technology (see e.g. Bourque, (1995), Plant Science 105, 125-149); sense regulation [co-suppression] (see e.g. Zhang et al., (1992) The Plant Cell 4, 1575-1588). Further options for down regulation of gene expression include the use of ribozymes, e.g. hammerhead ribozymes, which can catalyse the site-specific cleavage of RNA, such as mRNA (see e.g. Jaeger (1997) “The new world of ribozymes” Curr Opin Struct Biol 7:324-335. Nucleic acids and associated methodologies for carrying out down-regulation (e.g. complementary sequences) form one part of the present invention. As is well known to those skilled in the art, Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245).

The present invention also encompasses the expression product of any of the functional nucleic acid sequences disclosed above, plus also methods of making the expression product by expression from encoding nucleic acid therefore under suitable conditions, which may be in suitable host cells.

Following expression, the recombinant product may, if required, be isolated from the expression system. Generally however the polypeptides of the present invention will be used in vivo (in particular in planta).

Modified Starches

The present inventors have confirmed the importance of SUSIBA2 in regulation of starch synthesis by use of antisense oligodeoxynucleotide (ODN) technology (Dagle, J. M. & Weeks, D. L., (2001) Differentiation 69, 75-82). The ectopic expression of iso1 and sbeIIb was followed in intersected sucrose-treated barley leaves after addition of an 18-mer antisense ODN against susiba2. RNA-gel blot analysis revealed that exposure to the antisense ODN suppressed iso1 and sbeIIb expression, providing functional evidence that SUSIBA2 is relevant for iso1 and sbeIIb activities.

Additionally the inventors investigated the effects of the antisense ODN technology on starch composition. The antisense susiba2 ODNs lead to a significant increase in iodine staining, which is indicative of fewer or shorter branches in the starch molecules, consistent with reduced sbeIIb and iso1 activities. Also the solubility properties of the starch appeared to be altered.

Thus as discussed hereinbefore, the present invention provides a method of modifying polysaccharide (e.g. starch) production in a plant, the method including the step of down-regulating the expression of SUSIBA2 in the plant. Most preferably the down regulation comprises use of an oligonucleotide (ODN) based technology e.g. comprises the step of introducing antisense ODN into the plant, which antisense ODN comprises all or part of the complementary SUSIBA2 sequence e.g. 5′-CGCGGGGGACATGGCCTT-3′ as described below. Preferred methodologies for reducing gene expression using oligonucleotides may be any of those reviewed by Dagle, J. M. & Weeks, D. L., (2001) supra.

In a further aspect of the present invention there is provided a polysaccharide generated (in vivo) by a process comprising manipulation of a SUSIBA2 transcription factor as described above. Also embraced is starch produced in the transformed plants and cells discussed above. Such starch is preferably derived from amylopectin but has any of a decreased, increased or otherwise altered degree of branching, with a corresponding alteration in properties e.g. solubility, swelling or ability to form a paste rather than a gel when heated in water. Commodities (e.g. foodstuffs) comprising such starches form a further aspect of the present invention.

Other commodities which may benefit from the modified starches of the present invention include biodegradable plastics; food-processing thickeners; starch coated films, papers & textiles; paint thickeners; mining explosives; pharmaceuticals and glues. The modified starches can be used analogously to prior art starches in these materials, in ways which are well known to those skilled in the respective technical fields.

SUSIBA2 Antibodies

Purified SUSIBA2 protein produced recombinantly by expression from encoding nucleic acid therefor, may be used to raise antibodies employing techniques which are standard in the art. Antibodies and polypeptides comprising antigen-binding fragments of antibodies form a further part of the present invention, and may be used in identifying homologues from other plant species.

Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest.

For instance, Western blotting techniques or immunoprecipitation may be used (Armitage et al, 1992, Nature 357: 80-82). Antibodies may be polyclonal or monoclonal.

Antibodies may be modified in a number of ways. Indeed the term “antibody” should be construed as covering any polypeptide having a binding domain with the required SUSIBA2 specificity. Thus, this term covers antibody fragments, derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of Chimaeric antibodies are described in EP-A-0120694 and EP-A-0125023. It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the Vl and VH domains of a single antibody; (iv) the dAb fragment (Ward, E. S. et al., Nature 341, 544-546 (1989) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P Holliger et al Proc. Natl. Acad. Sci. USA 90 6444-6448, 1993).

Candidate SUSIBA2 polypeptides may be screened using these antibodies—e.g. by screening the products of an expression library created using nucleic acid derived from an plant of interest, or the products of a purification process from a natural source. A polypeptide found to bind the antibody may be isolated and then may be subject to amino acid sequencing. Any suitable technique may be used to sequence the polypeptide either wholly or partially (for instance a fragment of the polypeptide may be sequenced). Amino acid sequence information may be used in obtaining nucleic acid encoding the polypeptide, for instance by designing one or more oligonucleotides (e.g. a degenerate pool of oligonucleotides) for use as probes or primers in hybridization to candidate nucleic acid, or by searching computer sequence databases, as discussed further below.

The invention will now be further described with reference to the following non-limiting Figures and Examples. Other embodiments of the invention will occur to those skilled in the art in the light of these.

### FIGURES

FIG. 1. Structure of the susiba2 cDNA from barley.

The 18-nucleotides long sequence complementary to the human IGF1 receptor gene is underlined. The amino acid sequence of the tryptic fragment obtained by microsequencing from the overproduced SUSIBA2 (see FIG. 3) is shown by an open box.

FIG. 2. Analysis of the SUSIBA2 Primary Structure.

The amino acid sequence of SUSIBA2 was aligned with those of the nine closest matching proteins from a BLAST search. Identical amino acids are shown in back boxes, and similar amino acids are shown in grey boxes. The WRKYGQK peptide stretch is shown in pink. Putative nuclear localization signals are indicated with an orange # sign under the sequences. Ser- and Thr-rich regions are underlined in red. The zinc-finger-like motifs in the two WRKY domains are indicated with green vertical arrows under the sequence. A. t. hyp, Arabidopsis hypothetical protein; A. t. put, Arabidopsis putative protein; A. t. unk, Arabidopsis unknown protein; A. t. wrky20, Arabidopsis WRKY transcription factor 20; H. v. SUSIBA2, barley SUSIBA2; I. b. SPF1, sweet potato SPF1; L. e. hyp, tomato hypothetical proteins; N. t. wrky, tobacoo WRKY protein; R. r. d-i, white broom drought-induced protein.

FIG. 3. Purification of SUSIBA2.

His-tagged SUSIBA2 was subjected to FPLC purification on a Ni affinity column following overexpression of the susiba2 cDNA construct in E. coli. Amino acid sequences obtained by microsequencing of tryptic fragments are indicated.

FIG. 4. Binding of SUSIBA2 to Restriction Fragments Containing the SP8a Element.

(A) A portion of the iso1 promoter and restriction fragments spanning the SP8a element and flanking sequences.

(B) EMSA using the different SP8a-containing restriction fragments incubated in the presence (+) or absence (−) of SUSIBA2. The DNA-SUSIBA2 complexes are indicated by arrows.

FIG. 5. DNase I Footprint of SUSIBA2 to the iso1 Promoter Region.

(A) The DNA fragment (−736 to −537) was end-labeled and applied to Maxam-Gilbert sequencing. The sequencing reaction was used as markers (M). DNAse I digestion was performed in presence (+) or absence (−) of SUSIBA2. Positions, relative to the transcriptional start site, in the iso1 promoter are shown on the left. The protected region (−603 to −545) is indicated by an open box. Regions corresponding the SURE-a, SURE-b, and SURE-c sequences are designated as a, b, and c, respectively.

(B) Alignments of sequences from the protected region with the SURE element from the potato patatin promoter (Grierson et al., 1994).

FIG. 6. Binding of SUSIBA2 to Oligonucleotides Containing the SP8a, SURE, or W box Elements.

(A) The sequence of the three oligonucleotides. The SP8a, SURE and W box elements are shown in bold.

(B) EMSA using the different oligonucleotides incubated in the presence (+) or absence (−) of SUSIBA2. The DNA-SUSIBA2 complexes are indicated by arrows.

FIG. 7. Binding Competition Assay for SUSIBA2 with Oligonucleotides Containing the SURE or W box elements.

EMSA using the SURE or W box oligonucleotides incubated in the presence (+) or absence (−) of SUSIBA2, and without (−) or with (SURE or W box) a 100-fold molar excess of non-labeled competitor oligonucleotides.

FIG. 8. Temporal Expression Profile for susiba2 and iso1.

(A) RNA gel blot analysis of susiba2 and iso1 gene activity during barley endosperm development. Total RNA was isolated from barley endosperm on different days after pollination (d.a.p.) and used for RNA gel blot analysis. The blots were hybridized with probes specific for susiba2, iso1, or paz1. The sizes for the hybridizing fragments were around 2.4 kb for the susiba2 probe, 2.8 kb for iso1 probe, and 1.5 kb for the paz1 probe.

(B) TLC analysis of endogenous sucrose levels in developing barley endosperm from different d. a. p. A sucrose extract from 0.2 mg dry weight of developing endosperm from each time point was applied to TLC analysis.

FIG. 9. Ectopic Expression of susiba2 and iso1 in Barley Leaves.

Barley leaves were treated with exogenous sucrose at the concentrations indicated. Total RNA was isolated and hybridized to probes specific for susiba2, iso1, rbcS, and rbcL.

FIG. 10. Effects of the SURE-b ODN Decoy on iso1 Expression in Transformed Barley Endosperm.

(A) Schematic representation of the iso1::gfp construct (upper panel) and the SURE-b ODN decoy (lower panel).

(B) Transient expression assays of GFP fluorescence in transformed barley endosperm cells. Barley endosperm cells were transformed by microprojectile bombardment in absence (−) or presence (+) of the ODN decoy. For cotransformation, the iso1::gfp construct and ODN decoy were mixed at a molar ratio of 1:100.

(C) Average number of fluorescent foci per plate from nine independent transformation events.

FIG. 11. Primary Structure Comparison between SUSIBA2, the Rice and Wheat SUSIBA2 Orthologs, and SPF1.

(A) Alignment of the amino acid sequences between the conserved WRKY domains of SUSIBA2, the Rice and Wheat SUSIBA2 Orthologs, and SPF1.

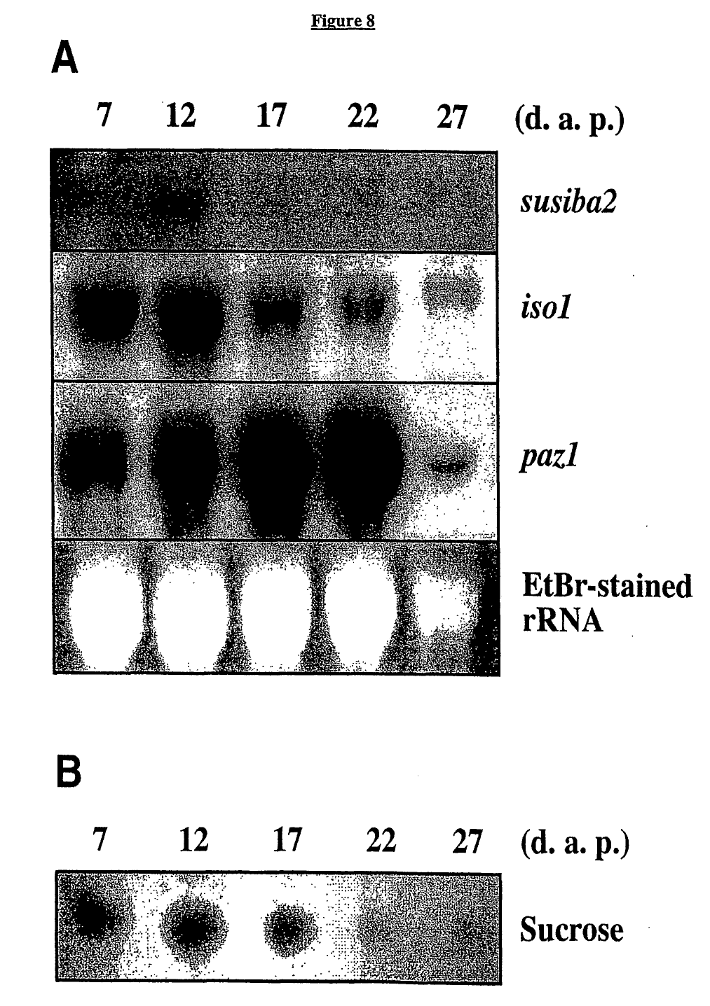

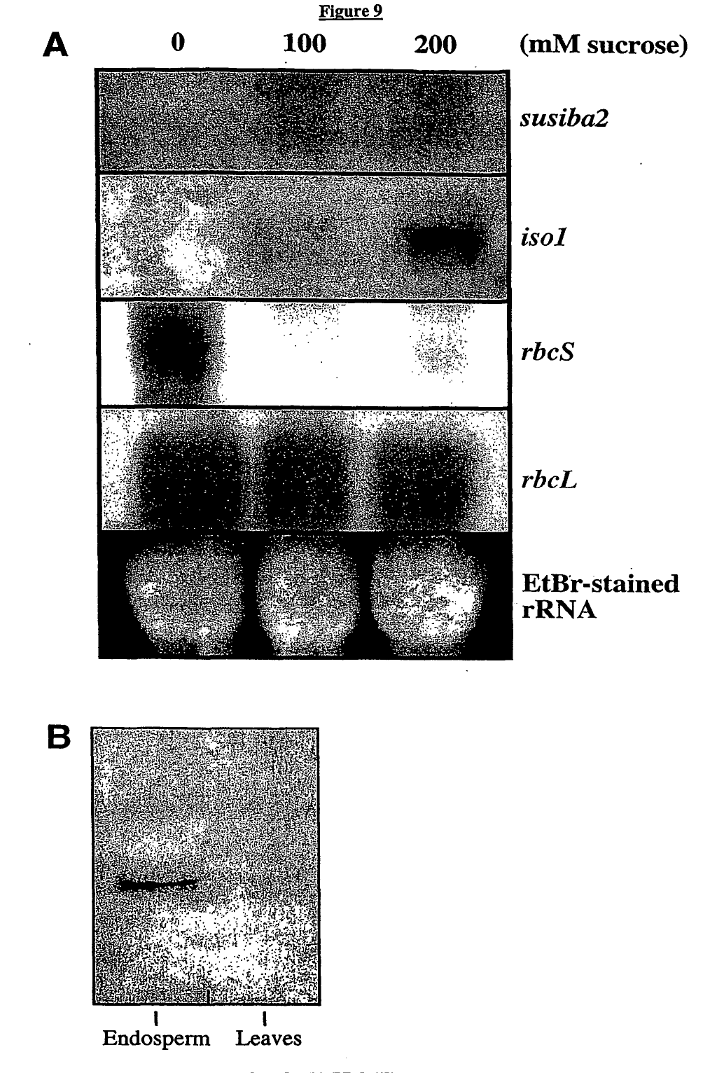

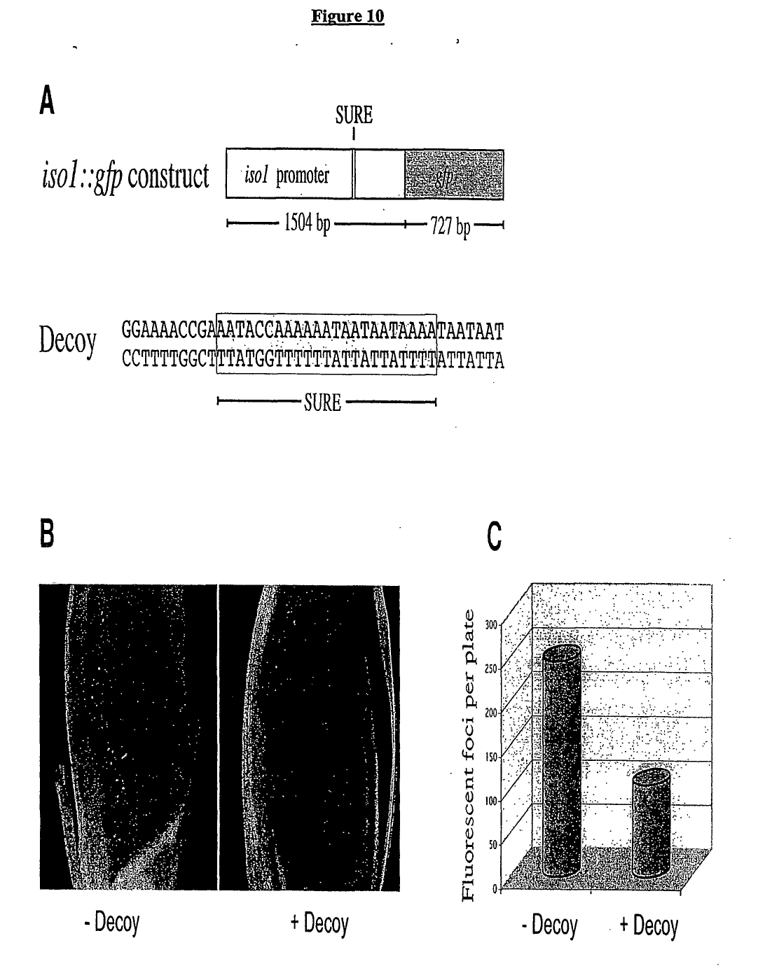

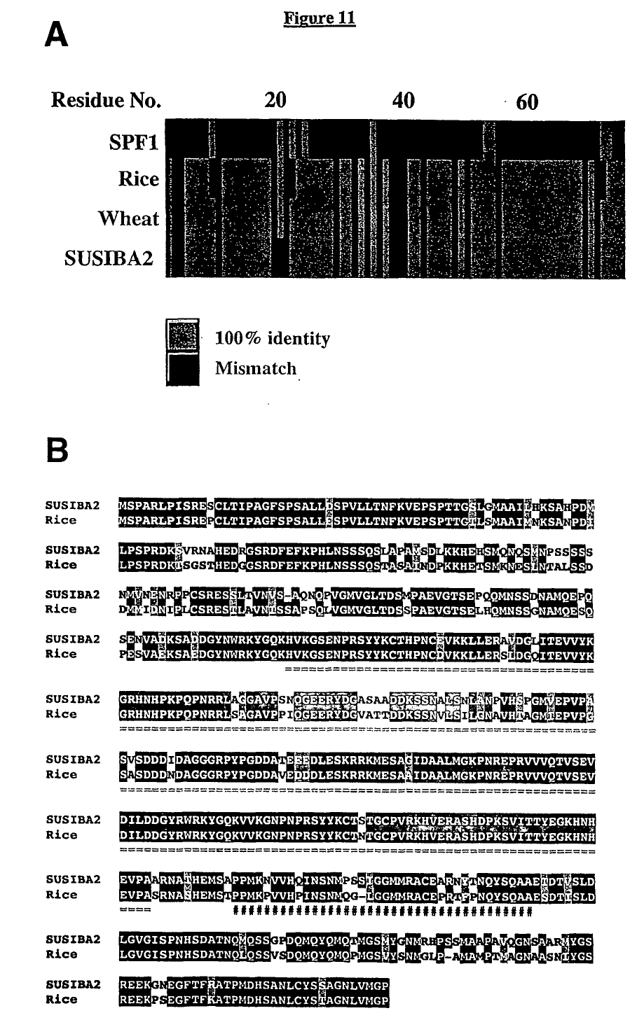

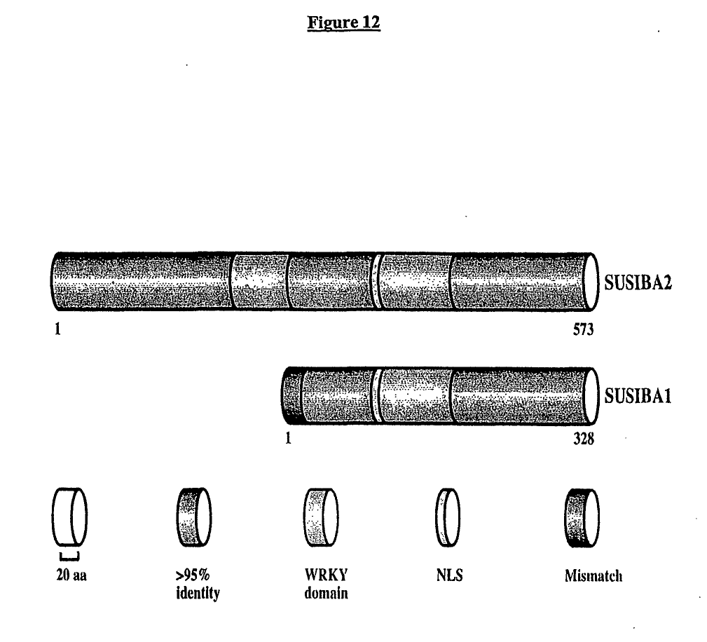

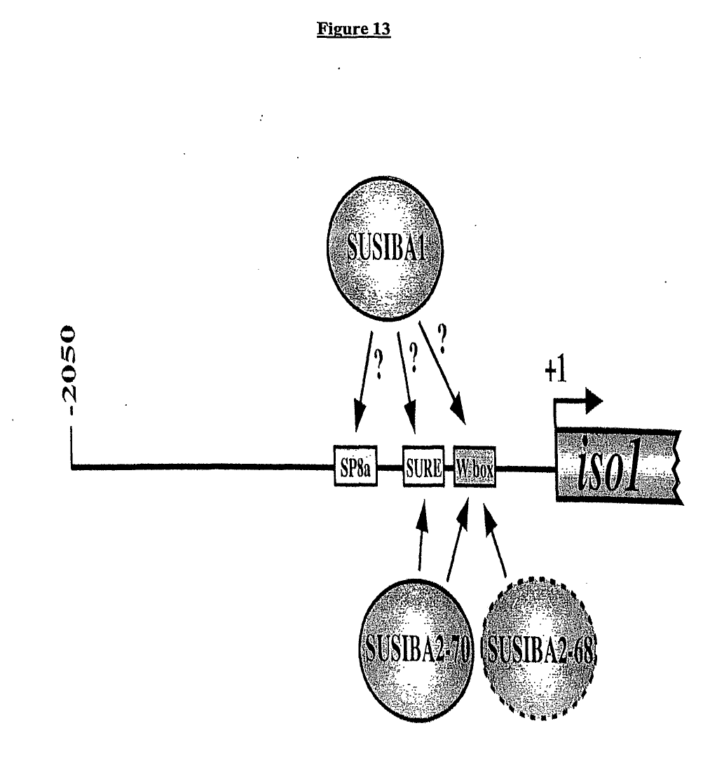

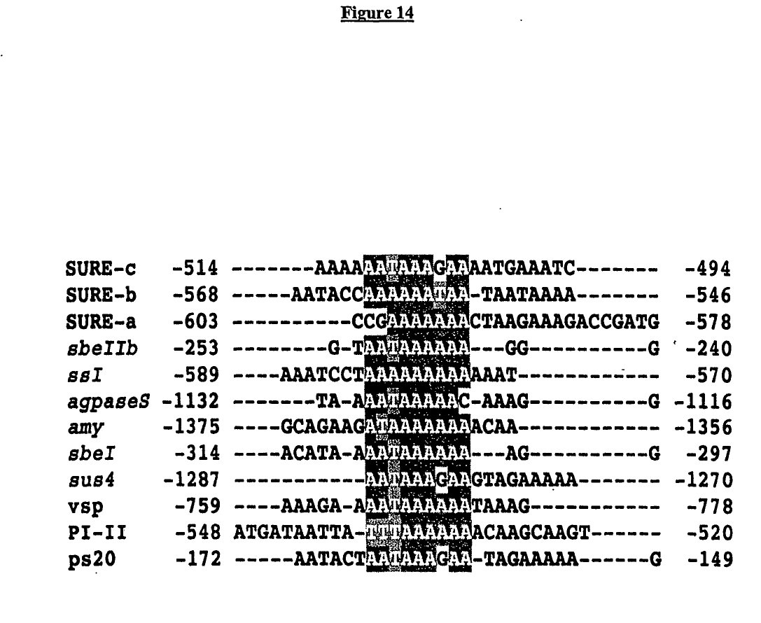

(B) Alignment of the entire amino acid sequence of SUSIBA2 with that of the rice SUSIBA2 ortholog. The portion corresponding to the isolated rice cDNA clone is denoted with a hatched double line under the sequences, with green indicating the aligned regions in (A). The “SUSIBA2-specific” sequence (c.f. FIG. 2) is indicated by a stretch of blue # signs.

FIG. 12. Schematic Comparison of SUSIBA2 and SUSIBA1.

SUSIBA1 and the C-terminal half of SUSIBA2 share >95% amino acid identity over most of their lengths. The exception is the divergent N-terminus of SUSIBA1. The conserved WRKY domains and the nuclear localization signals (NLS) are indicated.

FIG. 13. Model of the Interactions between the SUSIBAs and the iso1 Promoter.

SUSIBA2-70 binds to the SURE and W box elements, whereas SUSIBA2-68 binds only to the W box. Neither of the SUSIBA2 forms binds to the SP8a element. Note that we do not know whether SUSIBA2-68 exists in vivo.

FIG. 14. SURE and SURE-like sequences in plant promoters. Alignment of SURE sequences in promoters of plant genes known to be subjected to sugar induced expression. Selected A and T nucleotides are highlighted in red and green boxes, respectively. SURE-c, SURE-b, and SURE-a refer to the SURE sequences of barley iso1 (this work); sbeIIb, barley sbeIIb (Sun et al., 1998); ssI, barley ssI (Gubler et al., 2000); agpaseS, barley agpaseS (Thorbjørnsen et al., 2000); amy, Arabidopsis gene for β-amylase (Mita et al., 1995); sbeI, maize sbeI (Kim and Guiltinan, 1999); sus4, potato gene for sucrose synthase (Fu et al., 1995); vsp, soybean gene for vegetative storage protein (Rhee and Staswick, 1992); PI-II, potato gene for proteinase inhibitor II (Kim et al., 1991); ps20, potato patatin class I gene (Grierson et al., 1994).

### EXAMPLES

### Example 1

### Isolation and Characterization of the susiba2 cDNA

Primers were designed for RT-PCR amplification of barley cDNA using total RNA isolated from developing barley endosperm 9 days after pollination (d.a.p.). Primers combinaed information from sweet potato, cucumber and parsley transcription factor sequences (Ishiguro and Nakamura, 1994; Kim et al., 1997; Rushton et al., 1996). Surprisingly, one PCR product of an appropriate size was obtained (data not shown). This is particularly unexpected since given that, for instance, there are a large number (>70) of WRKY genes in Arabidopsis, and also that WRKY domains are closely conserved. The choice of developing endosperms may have contributed to there only being a single PCR product.

The amplified product was used as a probe to screen a 10 d.a.p. endosperm cDNA library and a leaf library. From screening of 1×10⁶ p.f.u. 14 positive clones were recovered. All positive clones were subjected to subcloning, restriction mapping and sequencing analysis.

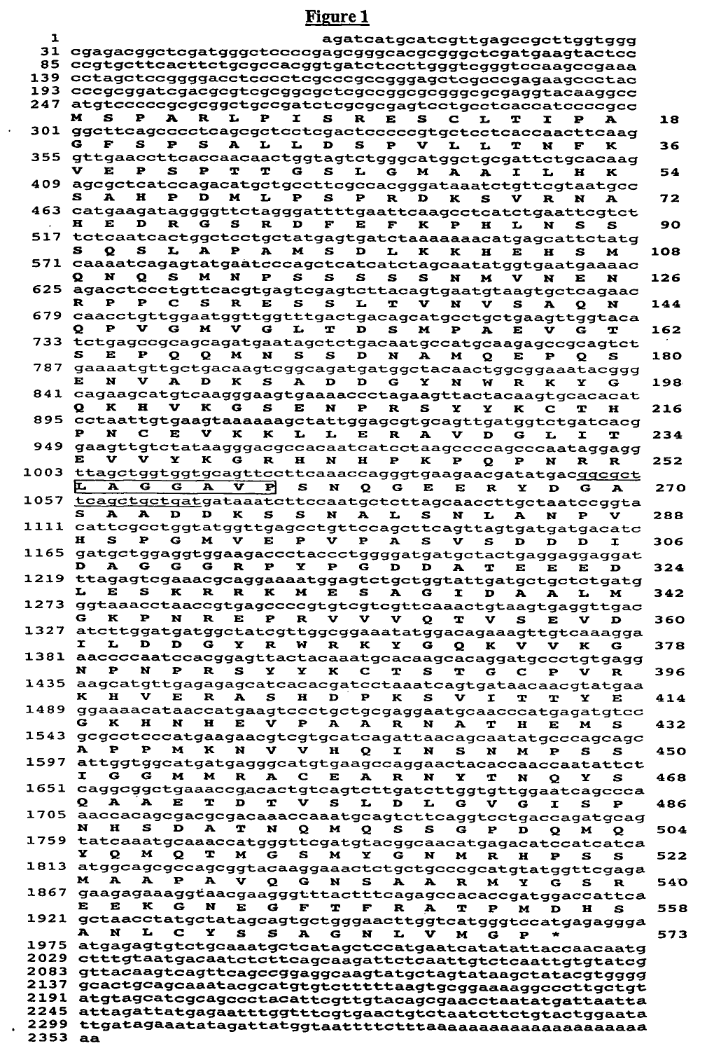

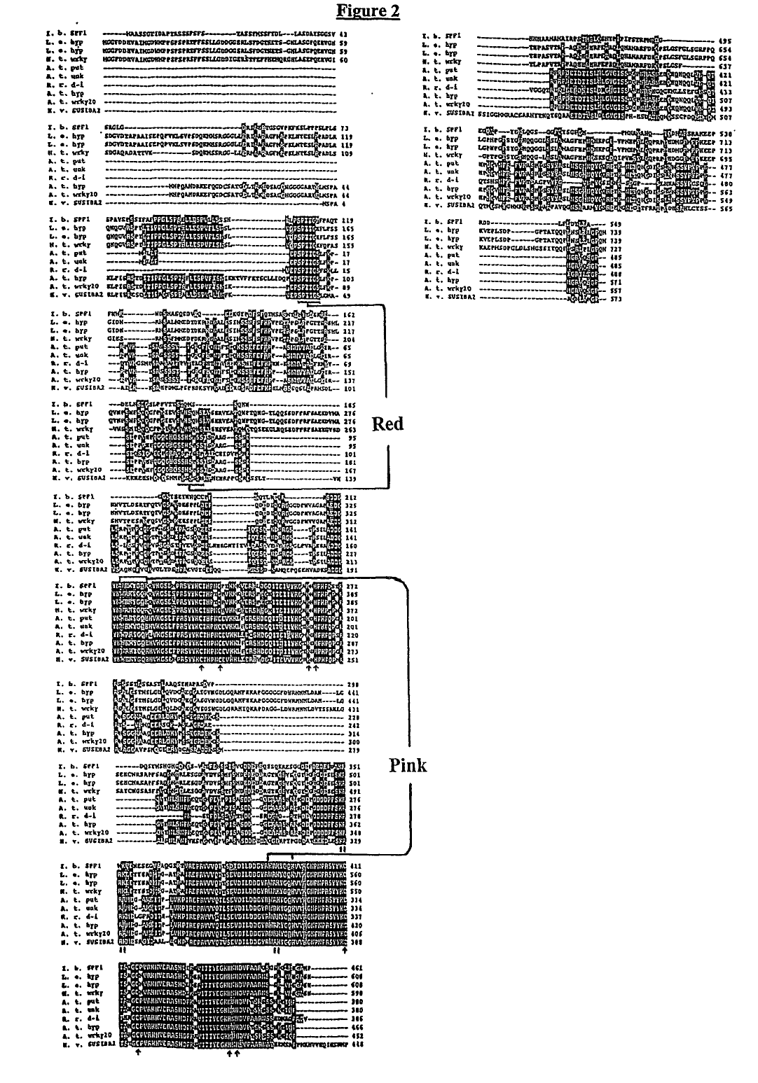

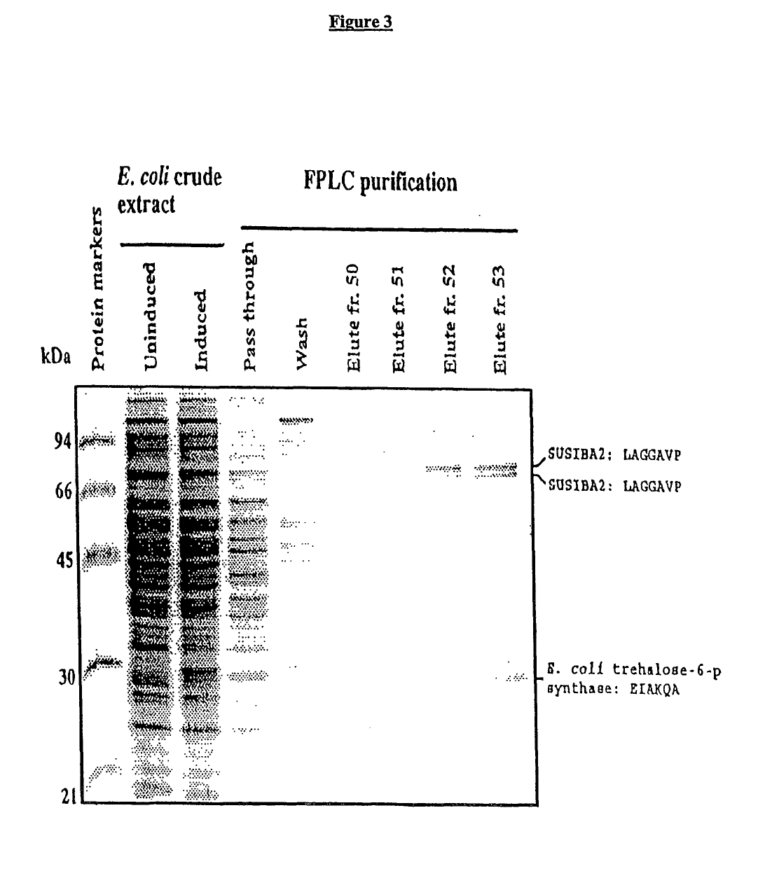

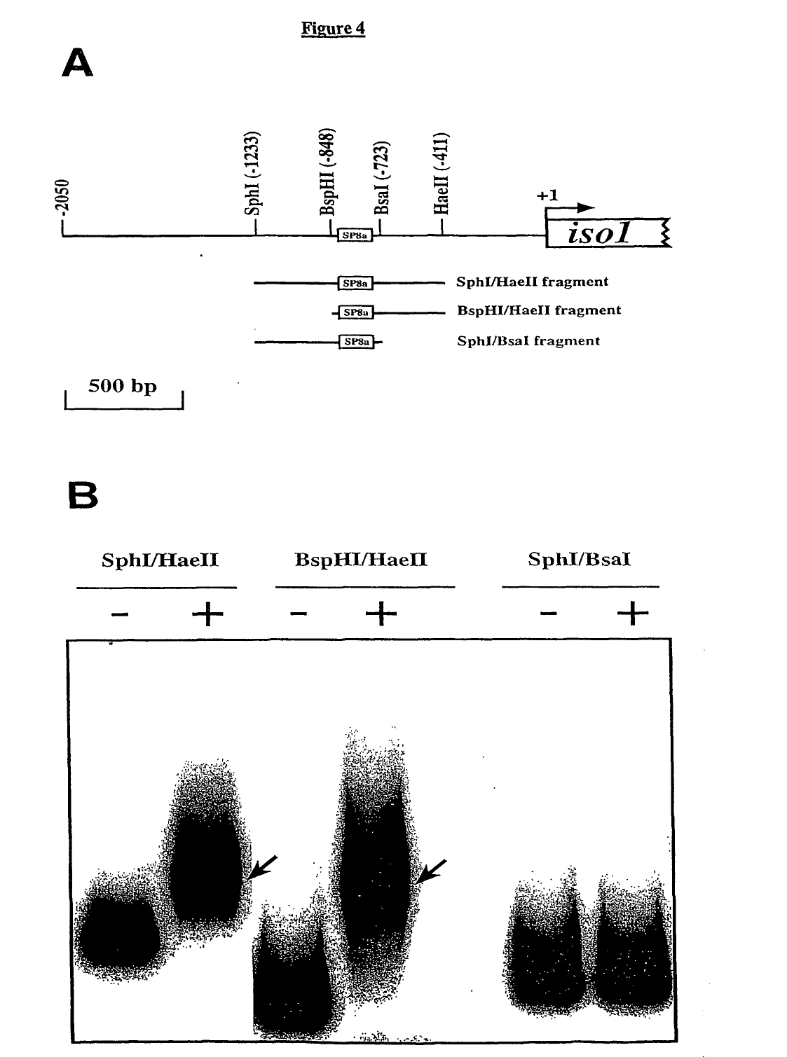

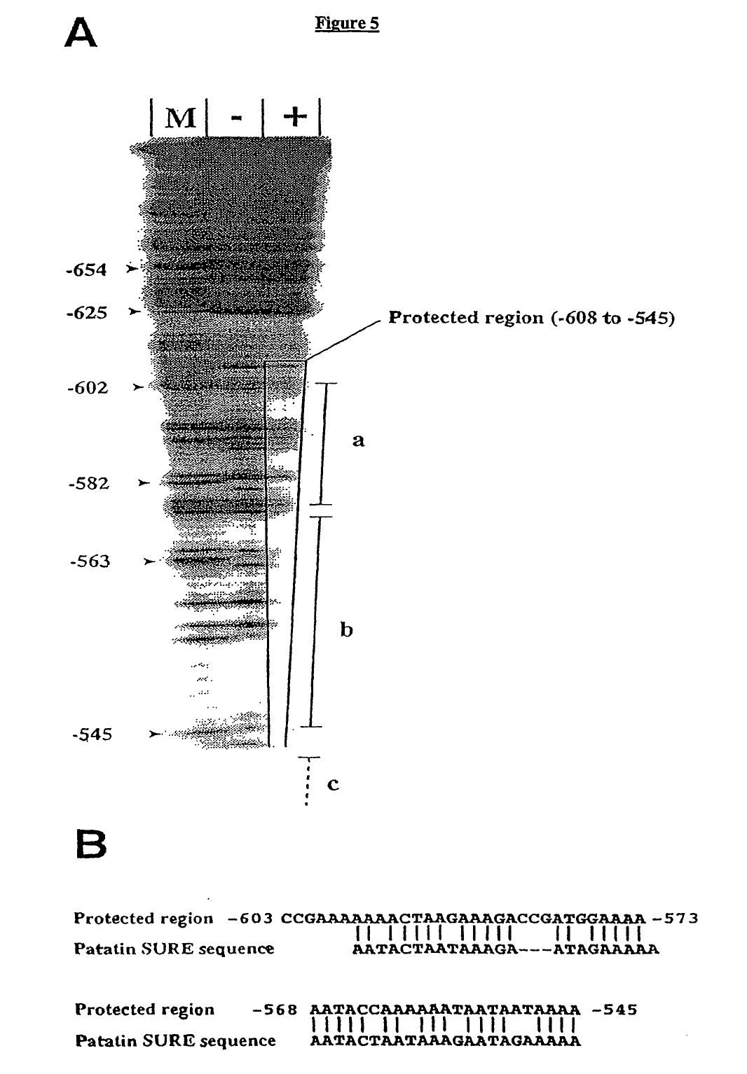

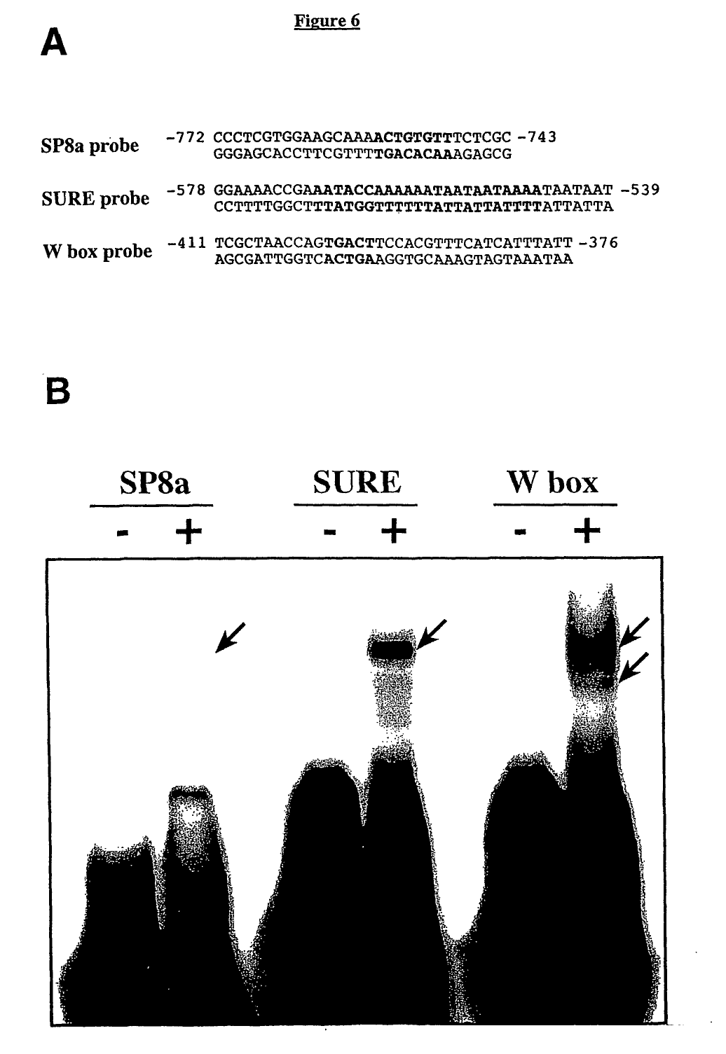

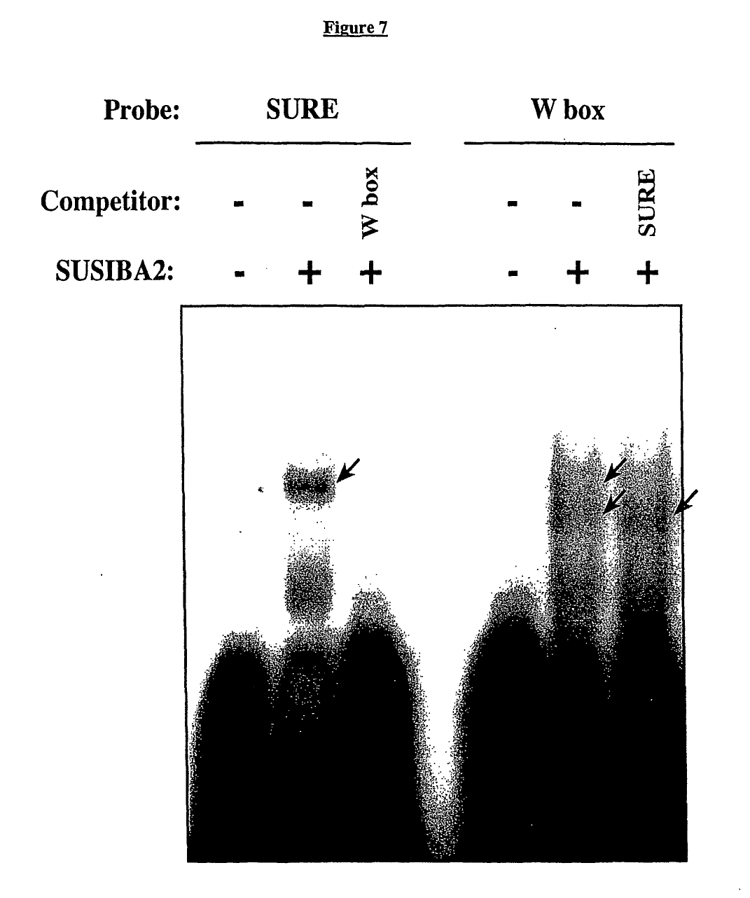

One clone was completely sequenced on both DNA strands. The length of the susiba2 cDNA is 2355 nucleotides, and the open reading frame starts at position 247 and ends at position 1966 (FIG. 1).

The cDNA encodes a 573-amino acids long polypeptide, SUSIBA2, with a calculated molecular mass of 62.2 kDa, and it has 5′ and 3′-untranslated regions and a poly (A⁺) tail sequences. These findings, together with the results from RNA gel blot analyses (see below), suggest that the susiba2 cDNA is full-length. One interesting feature of susiba2 is that an 18-nucleotides long sequence between nucleotides 1051 and 1068 (FIG. 1) is 100% complementary to the gene for human insulin-like growth factor 1 (IGF1) receptor (nucleotides 173-190).

The deduced amino acid sequence of SUSIBA2 was used in a BLAST search of the NCBI databases. Alignment of the 9 most top scoring matches with the SUSIBA2 sequence demonstrated that all ten proteins share a high degree of similarity and that SUSIBA2 belongs to group 1 of the WRKY superfamily of plant transcription factors (FIG. 2).

As would be expected from transcription factors, WRKY proteins contain nuclear localization signals (NLSs). The two most common NLSs are monopartite signals, which are short stretches enriched in basic amino acids, and bipartite signals, which are composed of two short basic stretches separated by a spacer (Merkle, 2001). The monopartite NLS depicted for WRKY proteins is conserved in SUSIBA2 (amino acids 328-331; FIG. 2). However, a closer inspection of the sequences suggests that the monopartite NLS in SUSIBA2 and other WRKY proteins might also constitute a bipartite NLS close to the consensus, KR-(24-74)-R..RK, for early auxin-inducible proteins (Abel and Theologis, 1995). One putative bipartite NLS in SUSIBA2 is KR-(66)-RK (amino acids 328-397). The other possibility is KR-(37)-R.RK (amino acids 328-370) and involves the WRKYGQK sequence itself in the C-terminal WRKY domain. The function of the WRKYGQK heptapeptide is unknown and it cannot be ruled out that it participates in the nuclear localization process, although a bipartite NLS arrangement for the N-terminal WRKYGQK sequence is not found. The most distinguishing feature of SUSIBA2 relative the other WRKY proteins is the 37-amino acids long insertion following the C-terminal WRKY domain (amino acids 435-471; FIG. 2). A BLAST search with the insertion sequence did not show any matches in the NCBI databases (however, see below), suggesting that SUSIBA2 is a novel type of WRKY protein.

The two WRKY domains (Eulgem et al., 2000) with their zinc finger motifs are highly conserved in SUSIBA2. Also the N-terminal Ser- and Thr-rich regions that are present in many WRKY proteins are found in SUSIBA2. The function of these regions is not known but is likely to involve regulation. Ser- and Thr-rich regions are characteristic of activation domains in transcription factors. It is generally believed that the C-terminal WRKY domain mediates sequence-specific binding to the cognate DNA elements, whereas the N-terminal WRKY domain facilitates DNA binding or engages in protein-protein interactions (Eulgem et al., 2000). The zinc fingers in each WRKY domain might be involved in binding to either DNA or proteins.

The WRKY class of proteins seem to be specific for the plant kingdom and are best known for their participation in various stress responses and senescence (Eulgem et al., 2000). Our results demonstrate that their sphere of activities has to be expanded to also include carbohydrate anabolism. It is probable that the unique properties of SUSIBA2, as compared to other, hitherto known, WRKY proteins, are attributable to regions outside of the two WRKY domains, where the amino acid sequences are relatively divergent (FIG. 2).

### Example 2

### Overproduction of SUSIBA2 in E. coli

The susiba2 cDNA was inserted into a pET expression vector and overexpressed in E. coli after IPTG induction. The overproduced protein was purified by a one-step FPLC on a His-Tag affinity column. Results from a typical overproduction and purification procedure are shown in FIG. 3.

The purified polypeptides were used for microsequencing, activity characterization and antibody production.

The purified protein fraction contained three polypeptides. They were separated on SDS-PAGE and subjected to in situ trypsin digestion. The digested fragments from each polypeptide were separated on HPLC and applied to mass-spectrometry analysis. The upper two bands corresponded to polypeptides with molecular masses of around 70 kDa and 68 kDa, respectively, and with the identical sequence LAGGAVP. This sequence is found in the deduced amino acid sequence of SUSIBA2 (FIG. 1; amino acids 235-241). The lower band corresponded to a 28-kDa polypeptide with the sequence of EIAKQA. This sequence matches E. coli trehalose-6-p synthase (amino acids 234-239). This enzyme copurified with the SUSIBA2 polypeptides also during repeated runs on the Ni column. It is therefore conceivable that the copurification was due to an unspecific association between SUSIBA2 and E. coli trehalose 6-P synthase, which may reflect an interaction of biological significance between SUSIBA2 and barley trehalose 6-P synthase. A role for trehalose and trehalose-6-phosphate in plant sugar signaling has been proposed (Goddijn and Smeekens, 1998).

Thus purified SUSIBA2 appeared in two forms, SUSIBA2-70 and SUSIBA2-68, of approximately 70 and 68 kDa, respectively (FIG. 3). Possibly, SUSIBA2-68 is a truncated version of SUSIBA2-70. This notion is supported by preliminary results, which indicate that the level of SUSIBA-70 increases at the expense of SUSIBA2-68 with increasing concentrations of protease inhibitors.

Since the purified SUSIBA2 still contains the N-terminal His-Tag, it seems less likely that any truncation should be at the N-terminal as that would leave too few His on SUSIBA2-68 to bind to the Ni column. Alternatively, the size difference between SUSIBA2-70 and SUSIBA-68 is an effect of posttranslational modification. The two SUSIBA2 polypeptides were extracted and used for production of polyclonal antibodies.

### Example 3

### Characterization of SUSIBA2 DNA-Binding Activity

As an initial test of the DNA-binding activity of SUSIBA2, we performed electrophoresis mobility shift assays (EMSAs) with three overlapping nucleotide fragments from the barley iso1 promoter, all containing the SP8a sequence (FIG. 4).

The 822-bp long SphI/HaeII fragment, encompassing SP8a and flanking sequences, as well as the 437-bp long BspHI/HaeII fragment, containing SP8a and downstream flanking sequence, formed prominent DNA-protein complexes with the purified SUSIBA2 fraction. Much to our surprise, however, the 510-bp long SphI/BsaI fragment, containing SP8a and upstream flanking sequence, bound only very poorly to SUSIBA2. We concluded that, in fact, the target for SUSIBA2 in the iso1 promoter is not SP8a but one or more sites downstream of the SP8a element. From further analysis of the segment downstream of SP8a in the BspHI/HaeII fragment we found that it harbors the sequence AATACCAAAAAATAATAATAAAA (nucleotides −568 to −545, relative the transcriptinal start site), which shares a high degree of identity with the SURE (sugar responsive) element, first reported by Grierson et al. (1994) from work on the potato class 1 patatin promoter. To follow up on this observation, we carried out EMSAs with oligonucleotides consisting of the iso1 SURE sequence. SUSIBA2 exhibited strong binding activity with the SURE probe, whereas binding to the SP8a probe was negligible (FIG. 6).

Since SUSIBA2 is a WRKY protein, it would be expected also to bind to the W box, the highly conserved binding site for WRKY proteins (Eulgem et al., 2000). A sequence identical to the consensus sequence for the W box, TGACT (nucleotides −400 to −396), was located in the iso1 promoter, and the iso1 W box sequence was included in the binding assay. With the W box probe, two DNA-SUSIBA2 complexes could be discerned. Thus, the results supported our earlier unexpected finding that SUSIBA2 does not bind to the SP8a element, and showed that, instead, it interacts with the SURE and W box elements.

To further illustrate that SUSIBA2 binds to the SURE element in the iso1 promoter, we ran a DNaseI footprinting assay. A 210-bp long NruI/XbaI fragment, encompassing the SURE element, was incubated with purified SUSIBA2, followed by DNaseI digestion (FIG. 5).

As expected, the protected region covered the SURE element but also the upstream and downstream flanking sequences were protected. In searching for an explanation we found that two additional SURE-like sequences, one immediately upstream, CCGAAAAAAACTAAGAAAGACCGATG (nucleotides −603 to −578) and one immediately downstream, AAAAAATAAAGAAAATGAAATC (nucleotides −514 to −493), of the one located initially, are present in the iso1 promoter. We refer to these SURE sequences as SURE-a, SURE-b and SURE-c for the upstream, middle and downstream sequences, respectively. A conceivable scenario is that SUSIBA2 binds to all three SURE sequences, yielding a protected segment of >110 nucleotides. This would likely explain why the protection continued beyond the 3′ end of the restriction fragment (FIG. 5A). As is also suggested by the footprinting assay, the SUSIBA2-DNA interaction is stronger for SURE-b than for SURE-a. An attempt to carry out a footprinting assay with the 437-bp long BspHI/HaeII fragment (FIG. 4A), covering all three SURE sequences, produced poor quality results. This is not surprising since the recommended restriction fragment lengths for DNase I footprinting is 250 bp or less.

Having demonstrated that SUSIBA2 binds to the SURE and W box elements in the iso1 promoter, we were interested in studying the relationship between the two binding activities. To this end we performed a competition experiment where the same amount of SUSIBA2 was incubated with the SURE or W box probe (see FIG. 6), in the presence or absence of a 100-fold molar excess of non-labeled competitor. The EMSA revealed that the W box competitor could efficiently outcompete the SURE probe from SUSIBA2, and that the SURE competitor could outcompete the W box probe from the larger of the two SUSIBA2 complexes (FIG. 7). It is logical to assume that the large and small complex contains the SUSIBA2-70 and SUSIBA2-68 polypeptides, respectively. Therefore, we interpret these results to mean that SUSIBA2-70 binds to both the W box and the SURE element, whereas SUSIBA2-68 binds only to the W box.

A model depicting the binding properties of SUSIBA2 with respect to the iso1 promoter is shown in FIG. 13. As has been demonstrated above, purified SUSIBA2 binds strongly to the SURE element and the W box but only poorly to the SP8a motif. Binding to the W box is consistent with SUSIBA2 being a WRKY protein, whereas binding to the SURE element displays a novel feature for a WRKY protein. The results herein suggest that the extreme C-terminal may be required for SURE-specific interactions. Another region that merits attention is the insertion in SUSIBA2 right after the C-terminal WRKY domain (FIGS. 2 and 11).

### Example 4

### Expression of susiba2 and iso1 correlates with Sucrose Levels

In previous investigations we have shown that expression of the barley iso1 gene is sugar inducible (Sun et al., 1999). In light of the different reports pointing to the involvement of the SURE element in sugar signaling (Fu et al., 1995; Grierson et al., 1994; Kim and Guiltinan, 1999; Kim et al., 1991; Li et al, 2002; Mita et al., 1995; Rhee and Staswick, 1992), and the results here, showing the SURE-binding activity of the SUSIBA2 transcription factor, it was of interest to study the expression pattern of the susiba2 gene. For examination of the temporal expression, barley endosperms were collected 7 to 27 d.a.p. and total RNA was isolated. RNA gel blot analysis was performed with susiba2- or iso1-specific probes, or with a PCR-amplified probe for the paz1 gene, encoding the storage protein Z (Sørensen et al., 1989), which was included for comparison. The expression level for susiba2 was low compared to iso1 but the patterns were similar, with a peak at around 12 d.a.p. (FIG. 8A).

A similar time course was obtained also for the barley sbeIIa and sbeIIb genes (Sun et al., 1998). Expression of the paz1 gene increased up to or beyond 22 d.a.p. and then sharply declined, consistent with the results by Sørensen et al. (1989). To study the correlation between sucrose and susiba2 expression, we monitored the endogenous sucrose concentrations during endosperm development by TLC. The sucrose concentrations were determined in seeds from the same batches used for transcript analysis. A comparison between the temporal expression pattern for susiba2 and iso1 transcript accumulation and sucrose levels showed a very good correlation (FIG. 8B).

No susiba2 transcripts could be detected in normal barley leaves. However, ectopic susiba2 expression in leaves was achieved after supplying exogenous sugar (FIG. 9A). The SUSIBA2 which was produced in sugar-treated leaves where it could be localized to the nucleus (data not shown).

Ectopic expression of iso1, which agrees with previous results (Sun et al., 1999), was also achieved. As a comparison, the photosynthesis genes rbcS and rbcL, encoding, respectively, the small and large subunit of RUBISCO, were abundantly expressed in control leaves (0 mM sucrose). The nuclear rbcS was dramatically downregulated by sucrose while the plastidic rbcL was not notably affected. The expression studies were extended to the protein level, using the SUSIBA2 antibody in a protein gel blot analysis (FIG. 9B). The SUSIBA2 protein was found in endosperm but not in leaves.

The data from the expression analyses is consistent with a role for SUSIBA2 as a positive regulatory transcription factor for iso1 activity. Experimental evidence supporting this view comes from the antisense inhibition studies. They also suggest that the expression of the susiba2 gene itself is controlled via sugar signaling.

From previous work, we learned that SP8a in the iso1 promoter is a negative regulatory element that recruits a repressor in non-expressing organs, such as embryos and leaves (Sun et al., 1999). The data from the expression analyses here, and the affinity of SUSIBA2 for the SURE element, together strongly suggest that SUSIBA2 binds to the SURE element(s) in the iso1 promoter as a positive regulator.

### Example 5

### In vivo Analysis of Transformed Barley Endosperm Demonstrate the Relevance of the SURE Element for iso1 Promoter Activity

To experimentally verify the premise that the SURE-b serves as an activating element for iso1 expression, we carried out transient assays in which the transcription of a chimeric iso1-reporter construct was studied in transformed barley endosperm. In this experiment, the entire (1.5 kb) iso1 promoter was fused to the gfp reporter gene. The employment of the GFP reporter system for analyses of promoter activity has been successfully used in transformed barley embryos (Ahlandsberg et al., 1999, 2002b). Since the iso1 gene is not expressed in embryos (Sun et al., 1999), a protocol for transformation of barley endosperm was established allowing us to carry out transient assays with gfp reporter constructs also in transformed barley endosperm.

The activity of the iso1 promoter, manifested as GFP fluorescence, was followed in transformed barley endosperm in the presence or absence of a transcription factor oligodeoxynucleotide (ODN) decoy containing the SURE-b sequence (FIG. 10A).

The ODN decoy approach involves flooding the cells with enough double-stranded decoy to compete for binding of transcription factors with their consensus sequences in target genes. If present in high-enough concentrations, these decoys can negate the ability of the transcription factor to regulate gene expression. The transcription factor ODN technology is widely used in medical investigations (Mann and Dzau, 2000). Here, we choose to adopt the strategy for our studies on barley endosperm. As is demonstrated in FIG. 10B, the presence of the SURE-b decoy in the endosperm cells blocked most, if not all, iso1 promoter activity. As shown in this Figure, the expression of iso1 in transformed barley endosperm was mainly confined to the periphery of the endosperm, towards the aleurone layer. The significance of this finding, if any, is unclear. The endosperms used for transformation were from seeds 20 d.a.p., which is near maturity. Since programmed cell death of the starchy endosperm tissue in barley progresses from the center, the uneven distribution of iso1 activity might indicate that, at 20 d.a.p., only endosperm cells along the aleuron layer are metabolically viable. Alternatively, the distribution reflects a sugar gradient within the endosperm. The transfer cells, which facilitate transport of photosynthate to the endosperm, are located at the basal endosperm, suggesting that, on sucrose medium, the sugar concentration in the endosperm will be highest at the vicinity of the transfer cells (for a recent review on endosperm development, see Olsen, 2001).

The average effect of three independent experiments is shown in FIG. 10C.

### Example 6

### Isolation of cDNA for SUSABA2 Orthologs in Rice and Wheat

In the light of the disclosure above characterising the involvement of the SUSIBA2 transcription factor in regulation of iso1 expression in barley endosperm, it was plausible that SUSIBA2 orthologs should be found in sink tissues also in other plants. The primers used for cloning of the susiba2 cDNA were employed for RT-PCR amplification of total RNA isolated from rice and wheat endosperm. For both species, only one major RT-PCR product was obtained (data not shown). The PCR products were subcloned and sequenced.

The deduced amino acid sequences from the PCR products include the region between the two WRKY domains, and analysis of these segments from the rice and wheat sequences show that they share a high degree of identity between themselves and with SUSIBA2 (FIG. 11A).

It is notable, apart from the conserved WRKY domains, the overall degree of identity between SUSIBA2 and the SP8a-binding-SPF1 is rather low (28%; FIG. 11).

The entire sequence for the rice ortholog could be retrieved from the TIGR Rice Genome Project (http://www.tigr.org/tdb/e2k1/osa1/) on an unclassified entry with the temporary name 2017.t00012. Since the open reading frame for the rice clone could be obtained in its entirety, we were able to compare the complete primary sequence between SUSIBA2 and the rice ortholog (FIG. 11B). The two proteins are very similar, also in the extreme C- and N-termini (they share a high degree of amino acid identity (80%) along their entire lengths).

Notably, the “SUSIBA2-specific” C-terminal insertion (see Example 3) is present in the rice sequence.

Further investigations have identified SUSIBA2 orthologs also in potato and Arabidopsis (data not shown).

### Example 7

### Discussion of Relevance of SURE-Element Binding

The importance of the SURE cis element for sucrose induction of gene activity in plants was first demonstrated by Grierson et al. (1994) from work on the potato patatin promoter, and has been corroborated by a large number of studies in different plants (Fu et al., 1995; Kim and Guiltinan, 1999; Kim et al., 1991; Li et al, 2002; Mita et al., 1995; Rhee and Staswick, 1992). In some of these studies the assignment of the SURE sequence as a regulatory element was inferred from experimental data, i.e. binding assays, whereas in other cases it was based primarily on computational analyses. Here we show that two (and possibly three) SURE elements are located also in the barley iso1 promoter and that they provide binding sites for the SUSIBA2 transcription factor. Its presence in the barley iso1 and maize sbe1 promoter suggests that the SURE element should also be found in other starch synthesis genes subject to sugar induction. A database search of the proximal and distal promoter regions of sequences revealed that, indeed, SURE-like sequences are present in promoters of several genes encoding enzymes involved in starch synthesis. Among those are barley sbeIIb, and the barley genes for starch SSI (ssI) and the small subunit of AGPase (agpaseS).

Although all three SURE sequences in the barley iso1 promoter, as well as those identified in other genes, bear a good resemblance to the patatin SURE sequence in pair-wise comparisons, it is difficult to arrive at a consensus sequence for the SURE element. An alignment of a subset of reported SURE sequences is shown in FIG. 14.

The compilation also includes the SURE sequences from barley ssI and agpaseS. As can be seen, the SURE element is best described as an AT-rich box with the consensus core A/TAAANA, where N denotes any nucleotide. It is similar to the so-called A box response element in the wheat gbss1 promoter (Kluth et al., 2002). A/T-rich protein-binding sequences are common in both eukaryotic and prokaryotic genes, and certain protein motifs, such as SPKK, show preference for binding at A/T-rich sites (Churchill and Suzuki, 1989). A/T-rich regions might also be expected in some promoters since they facilitate DNA unwinding. The role of the A/T-rich box in the SURE element remains to be assessed. Certainly, DNA-protein interactions will be affected by the sequence context surrounding the A/T-rich region. This is illustrated by the presence of several A/TAAANA sequences in the DNase I hypersensitive region of the iso1 promoter upstream of −608 (FIG. 5).

We noted that, in contrast to the barley sbeIIb promoter, which contains a SURE element (FIG. 14), no SURE-like sequence was found in the promoter of the barley sbeIIa gene. Both sbeIIb and sbeIIa contain a W box (data not shown). We have reported previously (Sun et al., 1998) that expression of barley sbeIIb is endosperm specific while that of sbeIIa is not. We suggest that the W box in the iso1, sbeIIb and sbeIIa promoters serves as a general activating element while the sugar responsiveness conferred to iso1 and sbeIIb by the SURE elements contributes to their endosperm specificity (by the higher sugar levels in sink tissues as compared to embryos and vegetative tissues). Further control of endosperm specific expression is probably exerted via binding of repressor proteins in non-expressing tissues. From earlier work, we concluded that repressors are recruited to the Sp8a element in the barley iso1 promoter (Sun et al., 1999), and to the Bbl element in the second intron of barley sbeIIb (Ahlandsberg et al., 2002b).

The importance of the SURE element for iso1 expression is demonstrated by the decoy experiment (FIG. 10). The activity remaining in the presence of the SURE-b decoy could indicate that the decoy did not efficiently trap the SURE-binding activity. However, more likely, it illustrates that elements other than SURE are sufficient to maintain a basic level of iso1 activity. The involvement of the SURE element in regulation of iso1 expression is in line with the ectopic expression of iso1 in sugar-treated barley leaves (Sun et al., 1999). The emerging transcription factor ODN technology offers a powerful and convenient way to assess transcription factor function and involvement of cis elements both in vitro and in vivo. The strategy has gained rapid popularity in animal sciences, e.g. in studies on gene therapy (Mann and Dzau, 2000; Morishita et al., 1998). To our knowledge, the present report represents the first example of the transcription factor ODN technology in plant biology.

SUSIBA2 is the first transcription factor known to bind to a SURE element, and, in fact, it is the first transcription factor reported for regulation of starch synthesis. Although binding of nuclear protein fractions to the SURE element in the potato patatin (Grierson et al., 1994) and maize sbeI (Kim and Guiltinan, 1999) promoters, to the Sp8a sequence in the barley iso1 promoter (Sun et al., 1999), and to the Bbl element in the second intron of the barley sbeIIb gene (Ahlandsberg et al., 2002b) has been documented previously, the interacting trans factors have not been isolated or identified.

### Example 8

### Down-Regulation of Ectopically Expressed SUSIBA2

To confirm the importance of SUSIBA2 in regulation of starch synthesis, we adopted the antisense oligodeoxynucleotide (ODN) technology [Dagle, J. M. & Weeks, D. L. Differentiation 69, 75-82 (2001)]. The ectopic expression of iso1 and sbeIIb was followed in intersected sucrose-treated barley leaves after addition of an 18-mer antisense ODN against susiba2. RNA-gel blot analysis revealed that exposure to the antisense ODN suppressed iso1 and sbeIIb expression, providing functional evidence that SUSIBA2 is relevant for iso1 and sbeIIb activities. Expression of sbeIIa or rbcS, encoding the small subunit of the Calvin cycle enzyme RUBISCO, were not significantly affected. The results of the transient ODN transformation assay demonstrate the applicability of the antisense ODN technology on intact plant tissues.

Additionally the inventors investigated the effects of the antisense ODN technology on starch composition. The antisense susiba2 ODNs lead to a significant increase in iodine staining, which is indicative of fewer or shorter branches in the starch molecules, consistent with reduced sbeIIb and iso1 activities. Also the solubility properties of the starch were altered, as judged by increased turbidity (results not shown).

Methods used in Examples

Plant Material

Barley (Hordeum vulgare L. cv. Pongo), rice and wheat were grown in soil in a climate chamber under a 16/8 h photoperiod as described by Sun et al. (1998, 1999).

Oligonucleotides

The following oligonucleotide primers were used:

Primer 1,
5′-CCAAGAAGTTATTACAAGTG-3′
Primer 2,
5′-TGGTTATGTTTTCCTTCGTA-3′
Primer 3,
5′-GGAATTCCATATGTCCCCCGCGCGGCTGCC-3′
Primer 4,
5′-CGGATCCGGCTGAACTGACTTGTAAC-3′
Primer 5,
5′-CCCTCGTGGAAGCAAAACTGTGTTTCTCGC-3′
Primer 6,
5′-GCGAGAAACACAGTTTTGCTTCCACGAGGG-3′
Primer 7,
5′-GGAAAACCGAAATACCAAAAAATAATAATAAAATAATAAT-3′
Primer 8,
5′-ATTATTATTTTATTATTATTTTTTGGTATTTCGGTTTTCC-3′
Primer 9,
5′-TCGCTAACCAGTGACTTCCACGTTTCATCATTTATT-3′
Primer 10,
5′-AATAAATGATGAAACGTGGAAGTCACTGGTTAGCGA-3′
Primer 11,
5′-ATGACTCGAGCAGATTTTGGATTGCTAATGA-3′
Primer 12,
5′-ATGACCATGGGCCACCTCGTGTTGGTTCTTCGT-3′

Computational Analyses

Searches of the NCBI databases were performed with the BLAST service (http://www.ncbi.nlm.nih.gov/BLAST/). Alignments of nucleotide and amino acid sequences were carried out using the Clustal W Service at the European Bioinformatics Institute (http://www.ebi.ac.uk/clustalw), except for alignment of sweet potato, cucumber and parsley SPF1 cDNA sequences, which was done with the MacVector® program (Accelrys Inc., USA), and alignment of the SURE element sequences, which were run on the T-Coffee server (http://www.ch.embnet.org/software/TCoffee.html). For presentation purposes, the ALN output from the Clustal W program was fed into the BOXSHADE server (http://www.ch.embnet.org/software/BOX_form.html.

Isolation of cDNA Clones

Primers 1 and 2 were constructed according to consensus sequences for the SPF1 and SPF1-like cDNA sequences. Total RNA was isolated from developing barley, rice and wheat endosperm according to Sun et al. (1999). First-strand cDNAs were produced as described (Frohman et al., 1988) using primer 2. RT-PCR was carried out according to standard protocols (Sambrook et al., 1989) using primers 1 and 2. The PCR products were subcloned and sequenced. The RT-PCR product from barley was used as a probe to screen a barley cDNA library from developing endosperm 10 days after pollination. Library construction and screening, subcloning, DNA gel-blot analysis and sequence analysis were as described (Sun et al., 1998, 1999).

Overproduction of SUSIBA2 in E. coli, Microsequencing, Antibody Production and Protein Gel Blot Analysis

Two primers were designed, primer 3, with an NdeI restriction site, and primer 4, with a BamHI restriction site. These primers were used for PCR amplification of the susiba2 cDNA from the isolated full-length cDNA clone. The amplified cDNA was inserted into the expression vector pET-15b (Novagen Inc., Germany) between the NdeI and BamHI sites. The constructed plasmid was transformed into E. coli strain BL 21 (DE3). Overexpression was carried out according to the manual provided by the manufacture, except the IPTG induction was performed at 37° C. for 2 hrs. The overproduced protein was purified by FPLC™ on a column with a Ni⁺ chelating resin. SDS-PAGE was run as described previously (Sun et al. 1997).

Trypsin digestion and microsequencing were done in collaboration with Amersham Pharmacia Biotech., Sweden. Antisera were produced by AgriSera AB, Sweden after immunizing rabbits with the Coomassie-stained SUSIBA-70 and -68 polypeptide bands excised from the SDS gel. Protein gel blot analysis was performed as before (Sun et al. 1997).

Electrophoresis Mobility Shift Assay (EMSA)

EMSAs were carried out essentially as described by Sun et al. (1999). The competitor probes were prepared by annealing different non-labeled oligonucleotides in 14 mM Tris-HCl (pH 8.0), 7 mM MgCl₂.

DNase I Footprinting

Restriction fragments of the iso1 promoter were singly end-labeled and used together with purified SUSIBA2 and the Sure Track Footpring Kit (Amersham Pharmacia Biotech, Sweden). All procedures were according to the manual provided by the manufacturer.

RNA Gel Blot Analysis

RNA isolation and RNA gel blot analysis were performed as described previously (Sun et al. 1998, 1999).

Sucrose Isolation and Analysis, and Exogenous Sucrose Induction

Sucrose isolation, TLC and sucrose induction of ectopic gene expression in barley leaves were carried out as described (Sun et al. 1998, 1999).

Transformation and GFP Assay of Barley Endosperm

The 1504-bp long promoter region of the iso1 gene was PCR-amplified using primers 11 and 12 and fused to the gfp plasmid pN1473GFP as described by Ahlandsberg et al. (1999, 2002a), yielding the construct p48. The ODN SURE-b decoy was generated by annealing primers 7 and 8 in 14 mM Tris-HCl (pH 8.0), 7 mM MgCl₂. Transformation of barley endosperm by microprojectile bombardment, using a DuPont PDS 1000 He Biolistic Delivery System (Bio-Rad Laboratories, Hercules, Calif., USA), and transient assay of GFP fluorescence were performed as described by Ahlandsberg et al. (2002a) with the following exceptions. Barley caryopses of 19-22 d.a.p were bisected longitudinally and placed endosperm side up on DG3B medium, containing Murashige and Skoog medium (Murashige and Skoog, 1962) supplemented with 100 g l⁻¹ sucrose, 1.0 mg l⁻¹ thiamine-HCl, 0.25 g l-1 myo-inositol, 1.0 g l⁻¹ casein hydrolase, and 0.69 g l⁻¹ proline solidified by 5.0 g l⁻¹ Gelrite (Duchefa, Haarlem, The Netherlands), for two hours prior bombardment. Six bisected caryopses were placed in the center of a 20×90 mm Petri dish with DG3B medium, 13 cm below the stopping screen, and bombarded once using a 7.6 MPa rupture disc. After bombardment, the caryopses were kept for 48 hours in darkness on plates before GFP fluorescence assays.

Accession Numbers

GenBank accession numbers referred to in this work are: Arabidopsis gene for β-amylase, S77076; Arabidopsis hypothetical protein, T08930; Arabidopsis WRKY transcription factor 20, Q93WV0; Arabidopsis putative protein, NP567752; Arabidopsis unknown protein, AAK76566; barley agpaseS, AJ239130; barley iso1, AF142589; barley sbeIIb, AF064563; barley ssI, AF234163; barley WRKY proteins implicated in drought tolerance, BM816210 and BM816211; E. coli trehalose-6-P synthase, S33584; human IGF1 receptor, NM_{—}000875; maize sbeI, AF072724; potato patatin class I gene, M18880; potato gene for proteinase inhibitor II, X04118; Potato gene for sucrose synthase, U24087; soybean gene for vegetative storage protein, M76980; sweet potato SPF1, S51529; tobacco WRKY protein, BAB61056; tomato hypothetical proteins, CAC36397 and CAC36402; white broom drought induced protein, AAL32033.

After the priority date of the present application, the following sequences of the present invention were deposited:

Hordeum vulgare SUSIBA2 (susiba2) mRNA, complete cds gi|34329336|gb|AY323206.1|[34329336]; Oryza sativa SUSIBA2-like protein mRNA, partial cds; gi|34329334|gb|AY324393.1|[34329334]; Triticum aestivum SUSIBA2-like protein mRNA, partial cds gi|34329332|gb|AY324392.1|[34329332].

SUSIBA2 [Hordeum vulgare] gi|34329337|gb|AAQ63880.1|[34329337];

SUSIBA2-like protein [Oryza sativa]

gi|34329335|gb|AAQ63879.1|[34329335];

SUSIBA2-like protein [Triticum aestivum]

gi|34329333|gb|AAQ63878.1|[34329333].

Antisense Oligodeoxynucleotide (ODN) Technology

Barley (Hordeum vulgare cv Pongo) plants were grown in soil in a climate chamber as described by Sun et al. (1998, 1999). The plants were used after 12-h darkness. Barley leaves were detached and incubated with 200 mM Suc solution in presence of 5 μM of antisense ODN or sense ODN. One 18-mer antisense ODN (oligonucleotide 1) against barley susiba2 cDNA sequences was used and three of sense ODN of the same length (oligonucleotides 2-4) were used as controls. The sequences of oligonucleotides 1-4 were as follows: 1, 5′-CGCGGGGGACATGGCCTT-3′; 2, 5′-AAGGCCATGTCCCCCGCG-3′; 3, 5′-CCAGACATGCTGCCTTCG-3′; 4, 5′-CCTGCTATGAGTGATCTA-3′. After 24 h-incubation in darkness, the barley leaves were harvested and stored at −80° C. until further use.

Sequence Annex

Rice susiba2 cDNA

CGTTCGCTTGATGGTCAGATTACTGAAGTGGTTTATAAAGGGCGTCACAA
TCACCCTAAGCCCCAACCCAATAGGAGGCTGTCTGCCGGTGCAGTTCCTC
CAATCCAGGGTGAAGAAAGATATGATGGTGTGGCAACTACTGATGACAAA
TCTTCAAATGTTCTTAGCATTCTTGGTAATGCAGTACATACAGCTGGTAT
GATTGAGCCTGTTCCAGGCTCAGCTAGTGATGATGACAATGATGCCGGAG
GAGGGAGACCTTACCCTGGAGATGATGCTGTTGAGGATGATGATTTAGAG
TCAAAACGAAGGAAAATGGAATCTGCTGCTATTGATGCTGCTTTGATGGG
CAAGCCTAACCGTGAGCCTCGTGTTGTAGTACAAACGGTTAGTGAGGTTG
ACATCTTGGATGATGGGTACCGCTGGCGCAAGTATGGCCAGAAAGTAGTT
AAAGGAAACCCCAATCCACGGAGTTACTACAAGTGCACAAATACAGGATG
CCCAGTCAGGAAGCATGTTGAGAGAGCATCACATGATCCAAAATCAGTCA
TAACAACATACGAAGG

(the rice ortholog sequence can be retrieved from the TIGR Rice Genome Project (http://www.tigr.org/tdb/e2k1/osa1/) on an unclassified entry with the temporary name 2017.t00012)

Wheat susiba2 cDNA

ACAAGTGCACACATCCTAATTGTGAAGTAAAAAAGCTATTGGAGCGTGCG
GTTGATGGTCTGATCACGGAAGTTGTCTATAAGGGGCGCCATAATCATCC
TAAGCCCCAGCCTAATAGGAGGTTAGCTGGTGGTGCAGTTCCTTCGAACC
AGGGTGAAGAACGATATGATGGTGCGGCAGCTGCTGATGATAAATCTTCC
AATGCTCTTAGCAACCTTGCTAATCCGGTAAATTCGCCTGGCATGGTTGA
GCCTGTTCCAGTTTCAGTTAGTGATGATGACATAGATGCTGGAGGTGGAA
GACCCTACCCTGGGGATGATGCTACAGAGGAGGAGGATTTAGAGTTGAAA
CGCAGGAAAATGGAGTCTGCAGGTATTGATGCTGCTCTGATGGGTAAACC
TAACCGTGAGCCCCGTGTTGTCGTTCAAACTGTAAGTGAGGTTGACATCT
TGGATGATGGGTATCGTTGGCGGAAATATGGACAGAAAGTTGTCAAAGGA
AACCCCAATCCACGGAGTTACTACAAATGCACAAGCACAGGATGCCCTGT
GAGGAAGCATGTTGAGAGAGCATCGCATGATCCTAAATCAGTGATAACAA
CGTACGAAGGAAAACATAACCA

Wheat susiba2 Peptide

PRSYYKCTHPNCEVKKLLERAVDGLITEVVYKGRHNHPKPQPNRRLAGGA
VPSNQGEERYDGAAAADDKSSNALSNLANPVNSPGMVEPVPVSVSDDDID
AGGGRPYPGDDATEEEDLELKRRKMESAGIDAALMGKPNREPRVVVQTVS
EVDILDDGYRWRKYGQKVVKGNPNPRSYYKCTSTGCPVRKHVERASHDPK
SVITTYE

### REFERENCES

Ahlandsberg, S. Sathish, P., Sun, C. and Jansson, C. (1999). Green fluorescent protein as a reporter system in the transformation of barley cultivars. Physiol. Plant. 107, 194-200.
Ahlandsberg, S. Sathish, P., Sun, C. and Jansson, C. (2002a). A set of useful monocotyledon transformation vectors. Biotech. Lett. 23, 1871-1875.
Ahlandsberg, S., Sun, C., and Jansson, C. (2002b). An intronic element directs endosperm-specific expression of the sbeIIb gene during barley seed development. Plant Cell Rep. 20, 864-868.
Abel, S., and Theologis, A. (1995). A polymorphic bipartite motif signals nuclear targeting of early auxin-inducible proteins related to PS-IAA4 from pea (Pisum sativum). Plant J. 8, 87-96.
Bae, J. M., and Liu J. R. (1997). Molecular cloning and characterization of two novel isoforms of the small subunit of ADP-glucose pyrophosphorylase from sweet potato. Mol. Gen. Genet. 254, 179-185.
Ball, S., van de Wal, M. H. B. J., and Visser, R. G. F. (1998). progress in understanding the synthesis of amylose. Trends Plant Sci. 3, 462-467.
Buléon, A., Colonna, P., Planchot, V. and Ball, S. (1998). Starch granules: structure and biosynthesis. Int. J. Biol. Macromol. 23, 85-112.
Carlson, M. (1999). Glucose repression in yeast. Curr. Opin. Microbiol. 2, 202-207.
Churchill, M. E. A., and Suzuki, M. (1989). ‘SPKK’ Motifs Prefer to Bind to DNA at A/T-rich Sites. EMBO J. 8, 4189-4195.
Eulgem, T., Rushton, P. J., Robatzek, S., and Somssich, I. E. (2000). The WRKY superfamily of plant transcription factors. Trends Plant Sci. 5, 199-206.
Frohman, M. A., Dush, M. K., Martin, G. R. (1988). Rapid production of full-length cDNAs from rare transcripts: Amplification using a single gene-specific oligonucleotide primer. Proc. Natl. Acad. Sci. USA 85, 8998-9002.
Fu, H., Kim, S. Y., and Park, W. D. (1995). High-level tuber expression and sucrose inducibility of a potato sus4 sucrose synthase gene require 5′ and 3′flanking sequences and the leader intron. Plant Cell 7, 1387-1394.
Giuliano, G., Pichersky, E., Malik, V. S., Timko, M. P., Scolnic, P. A., and Cashmore, A. R. (1988). An evolutionary conserved protein binding sequence upstream of a plant light-regulated gene. Proc. Natl. Acad. Sci. USA 85, 7089-7093.
Goddijn, O., and Smeekens, S. (1998). Sensing trehalose biosynthesis in plants. Plant J., 14, 143-146.
Grierson, C., Du, J.-S., de Torres Zabalau, M., Beggs, K., Smith, C., Holdsworth, M., and Bevan, M. W. (1994). Separate cis sequences and trans factors direct metabolic and developmental regulation of a potato tuber storage protein gene. Plant J. 5, 815-826.
Gubler, F., Li, Z., Fieg, S., Jacobsen, J. V., and Morell, M. K. (2000). Cloning and characterization of a starch synthase I gene (accession No. AF234163) from barley. Plant Physiol. 122, 1459.
Halford, N. G., and Hardie, D. G. (1998). SNF1-related protein kinases: Global regulators of carbon metabolism in plants? Plant Mol. Biol. 37, 735-748.
Hardie, D. G., Carling, D., and Carlson, M. (1998). The AMP-activated/SNF1 protein kinase subfamily: Metabolic sensors of the eukaryotic cell? Annu. Rev. Biochem. 67, 821-855.
Hattori, T., Nakagawa, S., and Nakamura, K. (1990). High-level expression of tuberous root storage protein genes of sweet potato in stems of plantlets grown in vitro on sucrose medium. Plant Mol. Biol. 14, 595-604.
Ishiguro, S., and Nakamura, K. (1992). The nuclear factor SP8BF binds to the 5′-upstream regions of three different genes coding for major proteins of sweet potato tuberous roots. Plant Mol. Biol. 18, 97-108.
Ishiguro, S., and Nakamura, K. (1994). Characterization of a cDNA encoding a novel DNA-binding protein, SPF1, that recognizes SP8 sequences in the 5′ upstream regions of genes coding for sporamin and β-amylase from sweet potato. Mol. Gen. Genet. 244, 563-571.
Khoshnoodi, J., Larsson, C.-T., Larsson, H., and Rask, L. (1998). Differential accumulation of Arabidopsis thaliana Sbe2.1 and Sbe2.2 transcripts in response to light. Plant Sci. 135, 183-193.
Kim, S.-R., Costa, M. A., and An, G. (1991). Sugar response element enhances wound response of potato proteinase inhibitor II promoter in transgenic tobacco. Plant Mol. Biol. 17, 973-983.
Kim, D.-J., Smith, S. M., and Leaver, C. J. (1997). A cDNA encoding a putative SPF1-type DNA-binding protein from cucumber. Gene 185, 265-269.
Kim, K.-N., and Guiltinan, M. J. (1999). Identification of cis-acting elements important for expression of the starch-branching enzyme I gene in maize endosperm. Plant Physiol. 121, 225-236.
Kluth, A., Sprunck, S., Becker, D., Lörz, H., and Lütticke, S. (2002). 5′ deletion of a gbsss1 promoter region from wheat leads to changes in tissue and developmental specificities. Plant Mol. Biol. 49, 669-682.
Kossman, J., Visser, R. G. F., Müller-Röber, B., Willmitzer, L., and Sonnewald, U. (1991). Cloning and expression analysis of a potato cDNA that encodes branching enzyme: evidence for coexpression of starch biosynthetic genes. Mol. Gen. Genet. 230, 39-44.
Li, X., Xing, J., Gianfagna, T. J., and Janes, H. W. (2002). Sucrose regulation of ADP-glucose pyrophosphorylase subunit genes transcript levels in leaves and fruits. Plant Sci. 162, 239-244
Maeo, K., Tomiya, T., Hayashi, K., Akaike, M., Morikami, A., Ishiguro, S., Nakamura, K. (2001). Sugar-responsible elements in the promoter of a gene for β-amylase of sweet potato. Plant Mol. Biol. 46, 627-637.
Mann, M. J., and Dzau, V. J. (2000). Therapeutic applications of transcription factor decoy oligonucleotides. J. Clin. Invest. 106, 1071-1075.
Merkle, T. (2001). Nuclear import and export of proteins in plants: a tool for the regulation of signaling. Planta 213, 499-517.
Mita, S., Suzuki-Fujii, K., and Nakamura, K. (1995). Sugar-inducible expression of a gene for β-amylase in Arabidopsis thaliana. Plant Physiol. 107, 895-904.
Morishita, R., Higaki, J., Tomita N., and Ogihara T. (1998). Application of transcription factor “decoy” strategy as means of gene therapy and study of gene expression in cardiovascular disease. Circ. Res. 82, 1023-1028.
Murashige, T., and Skoog, F. (1962). A revised medium for rapid growth and bio assays with tobacco tissue cultures. Physiol. Plant. 15, 473-497.
Müller-Röber, B. T., Kossman, J., Hannah, L. C., and Willmitzer, L. (1990). One of two different ADP-glucose pyrophosphorylase genes from potato responds strongly to elevated levels of sucrose. Mol. Gen. Genet. 224, 136-146.
Myers, A. M., Morell, M. K., James, M. G., and Ball, S. G. (2000). Recent progress toward understanding biosynthesis of the amylopectin crystal. Plant Physiol. 122, 989-997.
Nakamura, Y. (1996). Some properties of starch debranching enzymes and their possible role in amylopectin biosynthesis. Plant Sci. 121, 1-18.
Nakamura, Y. (2002). Towards a better understanding of the metabolic system for amylopectin biosynthesis in plants: rice endosperm as a model tissue. Plant Cell. Physiol. 43, 718-725.
Olsen, O.-A. (2001). Endosperm development: Cellularization and cell fate specification. Annu. Rev. Plant Physiol. Plant Mol. Biol. 52, 233-267.
Ozturk, Z. N., Talamé, V., Deyholos, M., Michalowski, C. B., Galbraith, D. W., Gozukirmizi, N., Tuberosa, R., and Bohnert, H. J. (2002). Monitoring large-scale changes in transcript abundance in drought- and salt-stressed barley. Plant Mol. Biol. 48, 551-573.
Rhee, Y., and Staswick, P. E. (1992). Nucleotide sequence of a soybean vegetative storage protein vspB gene. Plant Physiol. 98, 794-795.
Rolland, F., Moore, B., and Sheen, J. (2002). Sugar sensing and signaling in plants. Plant Cell S185-S205.
Rook, F., Corke, F., Card, R., Munz, G., Smith, C., and Bevan, M. W. (2001). Impaired sucrose-induction mutants reveal the modulation of sugar-induced starch biosynthetic expression by abscisic acid signaling. Plant J. 26, 421-433.
Rushton, P. J., Torres, J. T., Parniske, M., Wernert, P., Hahlbrock, K., and Somssich, I. E. (1996). Interaction of elicitor-induced DNA-binding proteins with elicitor response elements in the promoters of parsley PR1 genes. EMBO J. 15, 5690-5700.
Sambrook, J., Fritsch, E. F., and Maniatis, T. (1989). Molecular Cloning. New York, Cold Spring Harbor Laboratory Press.
Sheen, J., Zhou, L., and Jang, J. C. (1999). Sugars as signaling molecules. Curr. Opin. Plant Biol, 2, 410-418.
Smeekens, S. (2000). Sugar-induced signal transduction in plants. Annu. Rev. Palnt Physiol. Plant Mol. Biol. 51, 49-81.
Smith, A. M. (2001). The biosynthesis of the starch granule. Biomacromol. 2, 335-341.
Sun, C., Sathish, P., Ahlandsberg, S., Deiber, A., and Jansson, C. (1997) Identification of four starch-branching enzymes in barley endosperm: partial purification of forms I, IIa and IIb. New Phytol. 137, 215-222.
Sun, C., Sathish, P., Ahlandsberg, S., and Jansson, C. (1998). The two genes. encoding starch-branching enzyme IIa and IIb are differentially expressed in barley. Plant Physiol 118, 37-49.
Sun, C., Sathish, P., Ahlandsberg, S., and Jansson, C. (1999). Analyses of isoamylase gene activity in wild-type barley indicate its involvement in starch synthesis. Plant Mol. Biol. 40, 431-443.
Sørensen M. B., Cameron-Mills, V., and Brandt, A. (1989). Transcriptional and post-transcriptional regulation of gene expression in developing barley endosperm. Mol. Gen. Genet. 217, 195-201.
Thorbjørnsen, T., Villand, P., Ramstad, V. E., Kleczkowski, L. A., Olsen, O.-A., and Opsahl-Ferstad, H.-G. (2000). Nucleotide sequence of the ADP-glucose pyrophosphorylase promoter (accession No. AF239130) of barley a gene involved in endosperm starch formation. Plant Physiol. 122, 1459.
Visser, R. G. F., Stolte, A, and Jacobsen, E. (1991). Expression of a chimaeric granule-bound starch synthase-GUS gene in transgenic potato plants. Plant Mol. Biol. 17, 691-699.
Zourelidou, M., de Torres-Zabala, M., Smith, C., and Bevan, M. W. (2002). Storekeeper defines a new class of plant-specific DNA-binding proteins and is a putative regulator of patatin expression. Plant J. 30, 489-497.

1. An isolated nucleic acid which comprises a nucleotide sequence which encodes a sugar-signalling transcription factor which is capable of activating a promoter of a gene encoding an enzyme involved in the synthesis or deposition of starch.

2. A nucleic acid as claimed in claim 1 wherein the transcription factor is a WRKY protein which is capable of activating the promoter within a plant in response to sugar levels in the plant

3. A nucleic acid as claimed in claim 2 wherein the promoter comprises at least one SURE element and\or W box element to which the transcription factor binds

4. A nucleic acid as claimed in claim 3 wherein the promoter is selected from the group consisting of iso1, sbe1, sbeIIb, ssI, and agpaseS.

5. A nucleic acid as claimed in claim 1 wherein the nucleotide sequence is a susiba2 nucleotide sequence which:
(i) encodes the SUSIBA2 polypeptide given in FIG. 1, or
(ii) encodes a variant SUSIBA2 polypeptide which is a variant of the SUSIBA2 amino acid sequence given in FIG. 1 and which shares at least about 50%, 60%, 70%, 80% or 90% identity therewith.

6. A nucleic acid as claimed in claim 5 wherein the nucleotide sequence:
(i) consists of the barley susiba2 coding sequence given in FIG. 1 or one which is degeneratively equivalent thereto,
(ii) comprises a wheat or rice susiba2 coding sequence given in the Sequence Annex, or one which is degeneratively equivalent to either.

7. A nucleic acid as claimed in claim 5 wherein the susiba2 nucleotide sequence encodes a derivative of a susiba2 coding sequence selected from the group consisting of the barley susiba2 shown in FIG. 1 or a sequence which is degeneratively equivalent thereto, a wheat susiba2 coding sequence or a sequence which is degeneratively equivalent thereto, a rice susiba2 coding sequence or a sequence which is degeneratively equivalent thereto by way of addition, insertion, deletion or substitution of one or more codons.

8. A nucleic acid as claimed in claim 5 wherein the susiba2 nucleotide sequence consists of an allelic or other homologous or orthologous variant of the barley susiba2 coding sequence given in FIG. 1.

9. An isolated nucleic acid which comprises a nucleotide sequence which is the complement of the transcription factor-encoding nucleotide sequence of claim 5.

10. An isolated nucleic acid for use as a probe or primer, said nucleic acid having a distinctive sequence of at least about 16-24 nucleotides in length, which sequence is present in FIG. 1 or a sequence which is degeneratively equivalent thereto, or the complement of either.

11. An isolated nucleic acid as claimed in claim 10 wherein the distinctive sequence encodes all or part of the SUSIBA2-specific sequence:
ppmknvvhqinsnmpssiggmmracearnytnqysqaa.

12. A process for producing a nucleic acid as claimed in claim 7.

13. A method for identifying or cloning a nucleic acid as claimed in claim 8, which method employs a nucleic acid probe or primer having a distinctive sequence of at least about 16-24 nucleotides in length, which sequence is present in FIG. 1 or a sequence which is degeneratively equivalent thereto, or the complement of either.

14. A method as claimed in claim 13, which method comprises the steps of:
(a) providing a preparation of nucleic acid from a plant cell;
(b) providing said nucleic acid probe or primer,
(c) contacting nucleic acid in said preparation of step (a) with said probe or primer under conditions for hybridisation, and,
(d) identifying nucleic acid in said preparation which hybridises with said nucleic acid molecule.

15. A method as claimed in claim 13, which method comprises the steps of:
(a) providing a preparation of nucleic acid from a plant cell;
(b) providing a pair of nucleic acid molecule primers suitable for PCR,
(c) contacting nucleic acid in said preparation with said primers under conditions for performance of PCR,
(d) performing PCR and determining the presence or absence of amplified PCR product.

16. A recombinant vector which comprises the nucleic acid of claim 1.

17. A vector as claimed in claim 16 wherein the nucleic acid is operably linked to a promoter for transcription in a host cell, wherein the promoter is optionally an inducible promoter.

18. A vector as claimed in claim 16 which is a plant vector.

19. A method which comprises the step of introducing the vector of claim 16 into a host cell, and optionally causing or allowing recombination between the vector and the host cell genome such as to transform the host cell.

20. A host cell containing or transformed with a heterologous vector of claim 16.

21. A method for producing a transgenic plant, which method comprises the steps of:
(a) providing the host cell of claim 20,
(b) regenerating a plant from the transformed plant cell.

22. A transgenic plant which is obtainable by the method of claim 17, or which is a clone, or selfed or hybrid progeny or other descendant of said transgenic plant, which in each case includes a heterologous nucleic acid which comprises a nucleotide sequence encoding a sugar-signalling transcription factor which is capable of activating a promoter of a gene encoding an enzyme involved in the synthesis or deposition of starch.

23. A transgenic plant as claimed in claim 22 which is a seed crop plant.

24. A part of propagule from a plant as claimed in claim 22, which includes a heterologous nucleic acid which comprises a nucleotide sequence encoding a sugar-signalling transcription factor which is capable of activating a promoter of a gene encoding an enzyme involved in the synthesis or deposition of starch, said plant optionally being a seed crop plant.

25. An isolated polypeptide sugar-signalling transcription factor which is encoded by the nucleotide sequence of claim 1.

26. A polypeptide as claimed in claim 25 which is the SUSIBA2 polypeptide shown in FIG. 1.

27. (canceled)

28. A method for activating the promoter of a gene encoding an enzyme involved in the synthesis or deposition of starch in a plant,
wherein the promoter is activated by a sugar-signalling transcription factor,
which method comprises the step of causing or allowing expression of a heterologous nucleic acid as claimed in claim 1 within the cells of the plant, thereby expressing the transcription factor therein.

29. A method as claimed in claim 28 which is preceded by the earlier step of introduction of the heterologous nucleic acid into a cell of the plant or an ancestor thereof.

30. A method for modulating the activity of a promoter of a gene encoding an enzyme involved in the synthesis or deposition of starch in a plant,
wherein the promoter is activated by a sugar-signalling transcription factor,
which method comprises any of the following steps of:
(i) introducing all or part of a nucleic acid as claimed in claim 9 in the plant such as to reduce transcription factor expression by an antisense ODN mechanism;
(ii) causing or allowing transcription from part of a nucleic acid which comprises a nucleotide sequence encoding a sugar-signalling transcription factor which is capable of activating a promoter of a gene encoding an enzyme involved in the synthesis or deposition of starch such as to reduce transcription factor expression by co-suppression;
(iii) providing a nucleic acid encoding a ribozyme specific for a nucleic acid which comprising a sequence encoding a sugar-signalling transcription factor which is capable of activating a promoter of a gene encoding an enzyme involved in the synthesis or deposition of starch,
(iv) providing a double-stranded RNA which comprises an RNA sequence encoding part of a sugar-signalling polypeptide, which is optionally a siRNA duplex consisting of between 20 and 25 base pairs.

31. A method of producing modified starch anabolism activity in plant comprising use of a method of claim 28, and optionally recovering starch from the plant.

32. A method of binding, activating, or identifying a promoter which includes at least one SURE element and\or W box element, which method employs the step of contacting said promoter with a polypeptide of claim 25.

33. A method of investigating or confirming whether a cis promoter element is present in a plant transcription factor consensus sequence in a target gene promoter, the method comprising:
(i) observing the expression of a reporter gene operably linked to the promoter in a plant cell in which the transcription factor is present,
(ii) introducing into the plant cell a double stranded oligodeoxynucleotide (ODN) decoy corresponding to the promoter element into the cell,
(iii) observing the expression of the reporter gene in the presence of the ODN decoy,
wherein a reduction in expression from (i) to (iii) confirms that the plant transcription factor binds the promoter element.

34-37. (canceled)
